SEQUENCE LISTING (278 sequences)

SEQ ID NO: 1 - RNA, 16 nt, synthetic construct
  source 1..16: mol_type = other RNA
tgcggaaggatcatta

SEQ ID NO: 2 - RNA, 17 nt, synthetic construct
  source 1..17: mol_type = other RNA
ctgcggaaggatcatta

SEQ ID NO: 3 - RNA, 29 nt, synthetic construct
  source 1..29: mol_type = other RNA
aataaggattgacagattgagagctcttt

SEQ ID NO: 4 - RNA, 24 nt, synthetic construct
  source 1..24: mol_type = other RNA
gtcgagttgtttgggaatgcagct

SEQ ID NO: 5 - RNA, 28 nt, synthetic construct
  source 1..28: mol_type = other RNA
ataaggattgacagattgagagctcttt

SEQ ID NO: 6 - RNA, 17 nt, synthetic construct
  source 1..17: mol_type = other RNA
cgatggaagtttgaggc

SEQ ID NO: 7 - RNA, 18 nt, synthetic construct
  source 1..18: mol_type = other RNA
gttcttagttggtggagt

SEQ ID NO: 8 - RNA, 26 nt, synthetic construct
  source 1..26: mol_type = other RNA
aaggattgacagattgagagctcttt

SEQ ID NO: 9 - RNA, 32 nt, synthetic construct
  source 1..32: mol_type = other RNA
aataaggattgacagattgagagctctttctt

SEQ ID NO: 10 - RNA, 15 nt, synthetic construct
  source 1..15: mol_type = other RNA
gcggaaggatcatta

SEQ ID NO: 11 - RNA, 29 nt, synthetic construct
  source 1..29: mol_type = other RNA
ataaggattgacagattgagagctctttc

SEQ ID NO: 12 - RNA, 20 nt, synthetic construct
  source 1..20: mol_type = other RNA
attagtgacgcgcatgaatg

SEQ ID NO: 13 - RNA, 30 nt, synthetic construct
  source 1..30: mol_type = other RNA
aataaggattgacagattgagagctctttc

SEQ ID NO: 14 - RNA, 19 nt, synthetic construct
  source 1..19: mol_type = other RNA
ttagtgacgcgcatgaatg

SEQ ID NO: 15 - RNA, 22 nt, synthetic construct
  source 1..22: mol_type = other RNA
tagtttgacattgtgaaaagac

SEQ ID NO: 16 - RNA, 21 nt, synthetic construct
  source 1..21: mol_type = other RNA
aattagtgacgcgcatgaatg

SEQ ID NO: 17 - RNA, 19 nt, synthetic construct
  source 1..19: mol_type = other RNA
cgttcttagttggtggagt

SEQ ID NO: 18 - RNA, 28 nt, synthetic construct
  source 1..28: mol_type = other RNA
ttcgggtcttgtaattggaatgagtaca

SEQ ID NO: 19 - RNA, 30 nt, synthetic construct
  source 1..30: mol_type = other RNA
aaataaggattgacagattgagagctcttt

SEQ ID NO: 20 - RNA, 20 nt, synthetic construct
  source 1..20: mol_type = other RNA
aggggtgtatttattagata

SEQ ID NO: 21 - RNA, 28 nt, synthetic construct
  source 1..28: mol_type = other RNA
ctctagtgcagatcttggtggtagtagc

SEQ ID NO: 22 - RNA, 28 nt, synthetic construct
  source 1..28: mol_type = other RNA
gccggaaagttggtcaaacttggtcatt

SEQ ID NO: 23 - RNA, 27 nt, synthetic construct
  source 1..27: mol_type = other RNA
taaggattgacagattgagagctcttt

SEQ ID NO: 24 - RNA, 30 nt, synthetic construct
  source 1..30: mol_type = other RNA
tcgacgagtcgagttgtttgggaatgcagc

SEQ ID NO: 25 - RNA, 23 nt, synthetic construct
  source 1..23: mol_type = other RNA
tcgacgagtcgagttgtttggga

SEQ ID NO: 26 - RNA, 25 nt, synthetic construct
  source 1..25: mol_type = other RNA
taagggaagtcggcaaaatagatcc

SEQ ID NO: 27 - RNA, 24 nt, synthetic construct
  source 1..24: mol_type = other RNA
aaggattgacagattgagagctct

SEQ ID NO: 28 - RNA, 25 nt, synthetic construct
  source 1..25: mol_type = other RNA
ctctagtttgacattgtgaaaagac

SEQ ID NO: 29 - RNA, 27 nt, synthetic construct
  source 1..27: mol_type = other RNA
aataaggattgacagattgagagctct

SEQ ID NO: 30 - RNA, 26 nt, synthetic construct
  source 1..26: mol_type = other RNA
ataaggattgacagattgagagctct

SEQ ID NO: 31 - RNA, 17 nt, synthetic construct
  source 1..17: mol_type = other RNA
agcagaattcggtaagc

SEQ ID NO: 32 - RNA, 31 nt, synthetic construct
  source 1..31: mol_type = other RNA
aaataaggattgacagattgagagctctttc

SEQ ID NO: 33 - RNA, 26 nt, synthetic construct
  source 1..26: mol_type = other RNA
aggacgtcatagagggtgagaatccc

SEQ ID NO: 34 - RNA, 29 nt, synthetic construct
  source 1..29: mol_type = other RNA
aggtgaaattcttggatttgctgaagact

SEQ ID NO: 35 - RNA, 21 nt, synthetic construct
  source 1..21: mol_type = other RNA
aaggattgacagattgagagc

SEQ ID NO: 36 - RNA, 27 nt, synthetic construct
  source 1..27: mol_type = other RNA
cgagtcgagttgtttgggaatgcagct

SEQ ID NO: 37 - RNA, 32 nt, synthetic construct
  source 1..32: mol_type = other RNA
acaggacgtcatagagggtgagaatcccgtct

SEQ ID NO: 38 - RNA, 23 nt, synthetic construct
  source 1..23: mol_type = other RNA
ttagtgacgcgcatgaatggatt

SEQ ID NO: 39 - RNA, 20 nt, synthetic construct
  source 1..20: mol_type = other RNA
atcaactttcgatggtagga

SEQ ID NO: 40 - RNA, 23 nt, synthetic construct
  source 1..23: mol_type = other RNA
ttaacgaggaacaattggagggc

SEQ ID NO: 41 - RNA, 33 nt, synthetic construct
  source 1..33: mol_type = other RNA
tcgacgagtcgagttgtttgggaatgcagctct

SEQ ID NO: 42 - RNA, 21 nt, synthetic construct
  source 1..21: mol_type = other RNA
tatcaactttcgatggtagga

SEQ ID NO: 43 - RNA, 31 nt, synthetic construct
  source 1..31: mol_type = other RNA
aaaataaggattgacagattgagagctcttt

SEQ ID NO: 44 - RNA, 28 nt, synthetic construct
  source 1..28: mol_type = other RNA
ttgtcgaatcgcatggccttgcgctggc

SEQ ID NO: 45 - RNA, 27 nt, synthetic construct
  source 1..27: mol_type = other RNA
aggacgtcatagagggtgagaatcccg

SEQ ID NO: 46 - RNA, 23 nt, synthetic construct
  source 1..23: mol_type = other RNA
ctctagtttgacattgtgaaaag

SEQ ID NO: 47 - RNA, 19 nt, synthetic construct
  source 1..19: mol_type = other RNA
tgaggtaaagcgaatgatt

SEQ ID NO: 48 - RNA, 23 nt, synthetic construct
  source 1..23: mol_type = other RNA
tccttggaacaggacgtcataga

SEQ ID NO: 49 - RNA, 31 nt, synthetic construct
  source 1..31: mol_type = other RNA
caggacgtcatagagggtgagaatcccgtct

SEQ ID NO: 50 - RNA, 23 nt, synthetic construct
  source 1..23: mol_type = other RNA
ataaggattgacagattgagagc

SEQ ID NO: 51 - RNA, 22 nt, synthetic construct
  source 1..22: mol_type = other RNA
taaggattgacagattgagagc

SEQ ID NO: 52 - RNA, 25 nt, synthetic construct
  source 1..25: mol_type = other RNA
agaattcggtaagcgttggattgtt

SEQ ID NO: 53 - RNA, 24 nt, synthetic construct
  source 1..24: mol_type = other RNA
tctagtttgacattgtgaaaagac

SEQ ID NO: 54 - RNA, 31 nt, synthetic construct
  source 1..31: mol_type = other RNA
ttcgacgagtcgagttgtttgggaatgcagc

SEQ ID NO: 55 - RNA, 22 nt, synthetic construct
  source 1..22: mol_type = other RNA
taattagtgacgcgcatgaatg

SEQ ID NO: 56 - RNA, 23 nt, synthetic construct
  source 1..23: mol_type = other RNA
tcttcggatggatttgagtaaga

SEQ ID NO: 57 - RNA, 24 nt, synthetic construct
  source 1..24: mol_type = other RNA
aataaggattgacagattgagagc

SEQ ID NO: 58 - RNA, 18 nt, synthetic construct
  source 1..18: mol_type = other RNA
aagcagaattcggtaagc

SEQ ID NO: 59 - RNA, 21 nt, synthetic construct
  source 1..21: mol_type = other RNA
tatgaggtaaagcgaatgatt

SEQ ID NO: 60 - RNA, 22 nt, synthetic construct
  source 1..22: mol_type = other RNA
agtgaacgaaagttaggggatc

SEQ ID NO: 61 - RNA, 41 nt, synthetic construct
  source 1..41: mol_type = other RNA
tcgacgagtcgagttgtttgggaatgcagctctaagtgggt

SEQ ID NO: 62 - RNA, 32 nt, synthetic construct
  source 1..32: mol_type = other RNA
aaaataaggattgacagattgagagctctttc

SEQ ID NO: 63 - RNA, 44 nt, synthetic construct
  source 1..44: mol_type = other RNA
tcgacgagtcgagttgtttgggaatgcagctctaagtgggtggt

SEQ ID NO: 64 - RNA, 32 nt, synthetic construct
  source 1..32: mol_type = other RNA
aggtgaaattcttggatttgctgaagactaac

SEQ ID NO: 65 - RNA, 19 nt, synthetic construct
  source 1..19: mol_type = other RNA
atcgaagacgatcagatac

SEQ ID NO: 66 - RNA, 28 nt, synthetic construct
  source 1..28: mol_type = other RNA
acaggacgtcatagagggtgagaatccc

SEQ ID NO: 67 - RNA, 20 nt, synthetic construct
  source 1..20: mol_type = other RNA
aaggggtgtatttattagat

SEQ ID NO: 68 - RNA, 19 nt, synthetic construct
  source 1..19: mol_type = other RNA
aggggtgtatttattagat

SEQ ID NO: 69 - RNA, 25 nt, synthetic construct
  source 1..25: mol_type = other RNA
taaggattgacagattgagagctct

SEQ ID NO: 70 - RNA, 23 nt, synthetic construct
  source 1..23: mol_type = other RNA
aatgattaatagggatagtcggg

SEQ ID NO: 71 - RNA, 23 nt, synthetic construct
  source 1..23: mol_type = other RNA
aaggattgacagattgagagctc

SEQ ID NO: 72 - RNA, 24 nt, synthetic construct
  source 1..24: mol_type = other RNA
ttcgacgagtcgagttgtttggga

SEQ ID NO: 73 - RNA, 22 nt, synthetic construct
  source 1..22: mol_type = other RNA
aaggattgacagattgagagct

SEQ ID NO: 74 - RNA, 19 nt, synthetic construct
  source 1..19: mol_type = other RNA
gatggtaggatagtggcct

SEQ ID NO: 75 - RNA, 31 nt, synthetic construct
  source 1..31: mol_type = other RNA
tcgacgagtcgagttgtttgggaatgcagct

SEQ ID NO: 76 - RNA, 29 nt, synthetic construct
  source 1..29: mol_type = other RNA
taagttccttggaacaggacgtcatagag

SEQ ID NO: 77 - RNA, 18 nt, synthetic construct
  source 1..18: mol_type = other RNA
cgggaaaaggattggctc

SEQ ID NO: 78 - RNA, 22 nt, synthetic construct
  source 1..22: mol_type = other RNA
cgagtaggcaggcgtggaggtt

SEQ ID NO: 79 - RNA, 21 nt, synthetic construct
  source 1..21: mol_type = other RNA
ttgtttgggaatgcagctcta

SEQ ID NO: 80 - RNA, 22 nt, synthetic construct
  source 1..22: mol_type = other RNA
aataaggattgacagattgaga

SEQ ID NO: 81 - RNA, 20 nt, synthetic construct
  source 1..20: mol_type = other RNA
atgaggtaaagcgaatgatt

SEQ ID NO: 82 - RNA, 20 nt, synthetic construct
  source 1..20: mol_type = other RNA
tcgatggtaggatagtggcc

SEQ ID NO: 83 - RNA, 17 nt, synthetic construct
  source 1..17: mol_type = other RNA
agcgaatgattagaggc

SEQ ID NO: 84 - RNA, 28 nt, synthetic construct
  source 1..28: mol_type = other RNA
aaataaggattgacagattgagagctct

SEQ ID NO: 85 - RNA, 24 nt, synthetic construct
  source 1..24: mol_type = other RNA
ataaggattgacagattgagagct

SEQ ID NO: 86 - RNA, 19 nt, synthetic construct
  source 1..19: mol_type = other RNA
aactttcgatggtaggata

SEQ ID NO: 87 - RNA, 25 nt, synthetic construct
  source 1..25: mol_type = other RNA
ataaggattgacagattgagagctc

SEQ ID NO: 88 - RNA, 25 nt, synthetic construct
  source 1..25: mol_type = other RNA
gcgaatgattagaggccttggggtt

SEQ ID NO: 89 - RNA, 24 nt, synthetic construct
  source 1..24: mol_type = other RNA
gacgagtaggcaggcgtggaggtt

SEQ ID NO: 90 - RNA, 26 nt, synthetic construct
  source 1..26: mol_type = other RNA
tcgacgagtcgagttgtttgggaatg

SEQ ID NO: 91 - RNA, 23 nt, synthetic construct
  source 1..23: mol_type = other RNA
tttcgatggtaggatagtggcct

SEQ ID NO: 92 - RNA, 25 nt, synthetic construct
  source 1..25: mol_type = other RNA
gaaagttaggggatcgaagacgatc

SEQ ID NO: 93 - RNA, 21 nt, synthetic construct
  source 1..21: mol_type = other RNA
ttcgatggtaggatagtggcc

SEQ ID NO: 94 - RNA, 20 nt, synthetic construct
  source 1..20: mol_type = other RNA
cgtgcaaatcgatcgtcaaa

SEQ ID NO: 95 - RNA, 34 nt, synthetic construct
  source 1..34: mol_type = other RNA
gtcgagttgtttgggaatgcagctctaagtgggt

SEQ ID NO: 96 - RNA, 22 nt, synthetic construct
  source 1..22: mol_type = other RNA
agggaagtcggcaaaatagatc

SEQ ID NO: 97 - RNA, 18 nt, synthetic construct
  source 1..18: mol_type = other RNA
aggtgaaattcttggatt

SEQ ID NO: 98 - RNA, 22 nt, synthetic construct
  source 1..22: mol_type = other RNA
ttcgatggtaggatagtggcct

SEQ ID NO: 99 - RNA, 26 nt, synthetic construct
  source 1..26: mol_type = other RNA
aataaggattgacagattgagagctc

SEQ ID NO: 100 - RNA, 22 nt, synthetic construct
  source 1..22: mol_type = other RNA
tgaaaatggatggcgctcaagc

SEQ ID NO: 101 - RNA, 25 nt, synthetic construct
  source 1..25: mol_type = other RNA
aataaggattgacagattgagagct

SEQ ID NO: 102 - RNA, 21 nt, synthetic construct
  source 1..21: mol_type = other RNA
tcgatggtaggatagtggcct

SEQ ID NO: 103 - RNA, 22 nt, synthetic construct
  source 1..22: mol_type = other RNA
tttcgatggtaggatagtggcc

SEQ ID NO: 104 - RNA, 18 nt, synthetic construct
  source 1..18: mol_type = other RNA
actttcgatggtaggata

SEQ ID NO: 105 - RNA, 25 nt, synthetic construct
  source 1..25: mol_type = other RNA
tcgacgagtcgagttgtttgggaat

SEQ ID NO: 106 - RNA, 19 nt, synthetic construct
  source 1..19: mol_type = other RNA
gttcttagttggtggagtg

SEQ ID NO: 107 - RNA, 17 nt, synthetic construct
  source 1..17: mol_type = other RNA
aggtaaagcgaatgatt

SEQ ID NO: 108 - RNA, 21 nt, synthetic construct
  source 1..21: mol_type = other RNA
cgacgagtaggcaggcgtgga

SEQ ID NO: 109 - RNA, 25 nt, synthetic construct
  source 1..25: mol_type = other RNA
cgacgagtaggcaggcgtggaggtt

SEQ ID NO: 110 - RNA, 20 nt, synthetic construct
  source 1..20: mol_type = other RNA
ctttcgatggtaggatagtg

SEQ ID NO: 111 - RNA, 33 nt, synthetic construct
  source 1..33: mol_type = other RNA
acagcctctagtgcagatcttggtggtagtagc

SEQ ID NO: 112 - RNA, 22 nt, synthetic construct
  source 1..22: mol_type = other RNA
gttgtttgggaatgcagctcta

SEQ ID NO: 113 - RNA, 17 nt, synthetic construct
  source 1..17: mol_type = other RNA
agggataactggcttgt

SEQ ID NO: 114 - RNA, 33 nt, synthetic construct
  source 1..33: mol_type = other RNA
aggacgtcatagagggtgagaatcccgtctttg

SEQ ID NO: 115 - RNA, 21 nt, synthetic construct
  source 1..21: mol_type = other RNA
gttaggggatcgaagacgatc

SEQ ID NO: 116 - RNA, 33 nt, synthetic construct
  source 1..33: mol_type = other RNA
aaataaggattgacagattgagagctctttctt

SEQ ID NO: 117 - RNA, 27 nt, synthetic construct
  source 1..27: mol_type = other RNA
caggacgtcatagagggtgagaatccc

SEQ ID NO: 118 - RNA, 33 nt, synthetic construct
  source 1..33: mol_type = other RNA
agggaagcgcttgcaaccagactcggtcgtggg

SEQ ID NO: 119 - RNA, 21 nt, synthetic construct
  source 1..21: mol_type = other RNA
actttcgatggtaggatagtg

SEQ ID NO: 120 - RNA, 20 nt, synthetic construct
  source 1..20: mol_type = other RNA
taatagggatagtcgggggc

SEQ ID NO: 121 - RNA, 34 nt, synthetic construct
  source 1..34: mol_type = other RNA
aaagctcgtagttgaaccttgggtctggctggcc

SEQ ID NO: 122 - RNA, 27 nt, synthetic construct
  source 1..27: mol_type = other RNA
aacttagaactggtacggacaagggga

SEQ ID NO: 123 - RNA, 39 nt, synthetic construct
  source 1..39: mol_type = other RNA
tttgacctcggatcaggtaggagtacccgctgaacttaa

SEQ ID NO: 124 - RNA, 22 nt, synthetic construct
  source 1..22: mol_type = other RNA
attcggtaagcgttggattgtt

SEQ ID NO: 125 - RNA, 35 nt, synthetic construct
  source 1..35: mol_type = other RNA
tttgacctcggatcaggtaggagtacccgctgaac

SEQ ID NO: 126 - RNA, 39 nt, synthetic construct
  source 1..39: mol_type = other RNA
aacttagaactggtacggacaaggggaatctgactgtct

SEQ ID NO: 127 - RNA, 27 nt, synthetic construct
  source 1..27: mol_type = other RNA
aattcggtaagcgttggattgttcacc

SEQ ID NO: 128 - RNA, 34 nt, synthetic construct
  source 1..34: mol_type = other RNA
tttgacctcggatcaggtaggagtacccgctgaa

SEQ ID NO: 129 - RNA, 31 nt, synthetic construct
  source 1..31: mol_type = other RNA
tgatagaacaatgtagataagggaagtcggc

SEQ ID NO: 130 - RNA, 34 nt, synthetic construct
  source 1..34: mol_type = other RNA
tggcttcttagagggactatcggctcaagccgat

SEQ ID NO: 131 - RNA, 36 nt, synthetic construct
  source 1..36: mol_type = other RNA
gaactttgaggactgaagtggggaaaggttccatgt

SEQ ID NO: 132 - RNA, 28 nt, synthetic construct
  source 1..28: mol_type = other RNA
ttccggggggcatgcctgttcgagcgtc

SEQ ID NO: 133 - RNA, 28 nt, synthetic construct
  source 1..28: mol_type = other RNA
tttgacctcggatcaggtaggagtaccc

SEQ ID NO: 134 - RNA, 32 nt, synthetic construct
  source 1..32: mol_type = other RNA
gtacggtgaaactgcgaatggctcattaaatc

SEQ ID NO: 135 - RNA, 22 nt, synthetic construct
  source 1..22: mol_type = other RNA
ccactaatagggaacgtgagct

SEQ ID NO: 136 - RNA, 33 nt, synthetic construct
  source 1..33: mol_type = other RNA
gaactggtacggacaaggggaatctgactgtct

SEQ ID NO: 137 - RNA, 35 nt, synthetic construct
  source 1..35: mol_type = other RNA
tagaactggtacggacaaggggaatctgactgtct

SEQ ID NO: 138 - RNA, 22 nt, synthetic construct
  source 1..22: mol_type = other RNA
aacaatgtagataagggaagtc

SEQ ID NO: 139 - RNA, 32 nt, synthetic construct
  source 1..32: mol_type = other RNA
tttgacctcggatcaggtaggagtacccgctg

SEQ ID NO: 140 - RNA, 24 nt, synthetic construct
  source 1..24: mol_type = other RNA
cgatagcgcacaagtagagtgatc

SEQ ID NO: 141 - RNA, 26 nt, synthetic construct
  source 1..26: mol_type = other RNA
ggttctattttgttggtttctaggac

SEQ ID NO: 142 - RNA, 35 nt, synthetic construct
  source 1..35: mol_type = other RNA
ttagaactggtacggacaaggggaatctgactgtc

SEQ ID NO: 143 - RNA, 37 nt, synthetic construct
  source 1..37: mol_type = other RNA
acttagaactggtacggacaaggggaatctgactgtc

SEQ ID NO: 144 - RNA, 21 nt, synthetic construct
  source 1..21: mol_type = other RNA
caataaatactgatacagggc

SEQ ID NO: 145 - RNA, 36 nt, synthetic construct
  source 1..36: mol_type = other RNA
tacctggttgattctgccagtagtcatatgcttgtc

SEQ ID NO: 146 - RNA, 53 nt, synthetic construct
  source 1..53: mol_type = other RNA
tttgacctcggatcaggtaggagtacccgctgaacttaagcatatcaataagc

SEQ ID NO: 147 - RNA, 29 nt, synthetic construct
  source 1..29: mol_type = other RNA
ttcggtaagcgttggattgttcacccact

SEQ ID NO: 148 - RNA, 34 nt, synthetic construct
  source 1..34: mol_type = other RNA
agaactggtacggacaaggggaatctgactgtct

SEQ ID NO: 149 - RNA, 28 nt, synthetic construct
  source 1..28: mol_type = other RNA
ccaaggatgttttcattaatcagtgaac

SEQ ID NO: 150 - RNA, 47 nt, synthetic construct
  source 1..47: mol_type = other RNA
tttgacctcggatcaggtaggagtacccgctgaacttaagcatatca

SEQ ID NO: 151 - RNA, 29 nt, synthetic construct
  source 1..29: mol_type = other RNA
gaattcggtaagcgttggattgttcaccc

SEQ ID NO: 152 - RNA, 21 nt, synthetic construct
  source 1..21: mol_type = other RNA
gaataatagaataggacgtgt

SEQ ID NO: 153 - RNA, 32 nt, synthetic construct
  source 1..32: mol_type = other RNA
gtttgacctcggatcaggtaggagtacccgct

SEQ ID NO: 154 - RNA, 25 nt, synthetic construct
  source 1..25: mol_type = other RNA
gacaataaatactgatacagggctc

SEQ ID NO: 155 - RNA, 24 nt, synthetic construct
  source 1..24: mol_type = other RNA
atggaataatagaataggacgtgt

SEQ ID NO: 156 - RNA, 29 nt, synthetic construct
  source 1..29: mol_type = other RNA
aatcgatcgtcaaatttgggtataggggc

SEQ ID NO: 157 - RNA, 25 nt, synthetic construct
  source 1..25: mol_type = other RNA
cacccactaatagggaacgtgagct

SEQ ID NO: 158 - RNA, 24 nt, synthetic construct
  source 1..24: mol_type = other RNA
gtgcagatcttggtggtagtagca

SEQ ID NO: 159 - RNA, 30 nt, synthetic construct
  source 1..30: mol_type = other RNA
tttgacctcggatcaggtaggagtacccgc

SEQ ID NO: 160 - RNA, 34 nt, synthetic construct
  source 1..34: mol_type = other RNA
agttgatagaacaatgtagataagggaagtcggc

SEQ ID NO: 161 - RNA, 37 nt, synthetic construct
  source 1..37: mol_type = other RNA
gagaactttgaggactgaagtggggaaaggttccatg

SEQ ID NO: 162 - RNA, 30 nt, synthetic construct
  source 1..30: mol_type = other RNA
gatagaacaatgtagataagggaagtcggc

SEQ ID NO: 163 - RNA, 25 nt, synthetic construct
  source 1..25: mol_type = other RNA
tttgacctcggatcaggtaggagta

SEQ ID NO: 164 - RNA, 31 nt, synthetic construct
  source 1..31: mol_type = other RNA
tcgccaaggatgttttcattaatcagtgaac

SEQ ID NO: 165 - RNA, 26 nt, synthetic construct
  source 1..26: mol_type = other RNA
acttagaactggtacggacaagggga

SEQ ID NO: 166 - RNA, 38 nt, synthetic construct
  source 1..38: mol_type = other RNA
acttagaactggtacggacaaggggaatctgactgtct

SEQ ID NO: 167 - RNA, 25 nt, synthetic construct
  source 1..25: mol_type = other RNA
cggggggcatgcctgttcgagcgtc

SEQ ID NO: 168 - RNA, 31 nt, synthetic construct
  source 1..31: mol_type = other RNA
tacggtgaaactgcgaatggctcattaaatc

SEQ ID NO: 169 - RNA, 27 nt, synthetic construct
  source 1..27: mol_type = other RNA
aagtcggaatccgctaaggagtgtgta

SEQ ID NO: 170 - RNA, 44 nt, synthetic construct
  source 1..44: mol_type = other RNA
tttgacctcggatcaggtaggagtacccgctgaacttaagcata

SEQ ID NO: 171 - RNA, 22 nt, synthetic construct
  source 1..22: mol_type = other RNA
tgaaacttaaagaaattgacgg

SEQ ID NO: 172 - RNA, 23 nt, synthetic construct
  source 1..23: mol_type = other RNA
aggattggctctaagggtcgggc

SEQ ID NO: 173 - RNA, 16 nt, synthetic construct
  source 1..16: mol_type = other RNA
ctgttcgagcgtcatt

SEQ ID NO: 174 - RNA, 27 nt, synthetic construct
  source 1..27: mol_type = other RNA
gtgacaataaatactgatacagggctc

SEQ ID NO: 175 - RNA, 21 nt, synthetic construct
  source 1..21: mol_type = other RNA
atagcgacgttgctttttgat

SEQ ID NO: 176 - RNA, 24 nt, synthetic construct
  source 1..24: mol_type = other RNA
taagcgttggattgttcacccact

SEQ ID NO: 177 - RNA, 27 nt, synthetic construct
  source 1..27: mol_type = other RNA
tttttgatccttcgatgtcggctcttc

SEQ ID NO: 178 - RNA, 25 nt, synthetic construct
  source 1..25: mol_type = other RNA
gaccaacttagaactggtacggaca

SEQ ID NO: 179 - RNA, 28 nt, synthetic construct
  source 1..28: mol_type = other RNA
tgggacccgaaagatggtgaactatgcc

SEQ ID NO: 180 - RNA, 35 nt, synthetic construct
  source 1..35: mol_type = other RNA
tagttgatagaacaatgtagataagggaagtcggc

SEQ ID NO: 181 - RNA, 29 nt, synthetic construct
  source 1..29: mol_type = other RNA
aacttagaactggtacggacaaggggaat

SEQ ID NO: 182 - RNA, 22 nt, synthetic construct
  source 1..22: mol_type = other RNA
aagcggaggaaaagaaaccaac

SEQ ID NO: 183 - RNA, 26 nt, synthetic construct
  source 1..26: mol_type = other RNA
aaggatgttttcattaatcagtgaac

SEQ ID NO: 184 - RNA, 27 nt, synthetic construct
  source 1..27: mol_type = other RNA
gggacccgaaagatggtgaactatgcc

SEQ ID NO: 185 - RNA, 28 nt, synthetic construct
  source 1..28: mol_type = other RNA
atggatcgttactagtgggccatttttg

SEQ ID NO: 186 - RNA, 24 nt, synthetic construct
  source 1..24: mol_type = other RNA
acccactaatagggaacgtgagct

SEQ ID NO: 187 - RNA, 37 nt, synthetic construct
  source 1..37: mol_type = other RNA
agaactttgaggactgaagtggggaaaggttccatgt

SEQ ID NO: 188 - RNA, 24 nt, synthetic construct
  source 1..24: mol_type = other RNA
ataagcggaggaaaagaaaccaac

SEQ ID NO: 189 - RNA, 24 nt, synthetic construct
  source 1..24: mol_type = other RNA
tggtgaactatgcctgaatagggt

SEQ ID NO: 190 - RNA, 29 nt, synthetic construct
  source 1..29: mol_type = other RNA
atcgatcgtcaaatttgggtataggggcg

SEQ ID NO: 191 - RNA, 23 nt, synthetic construct
  source 1..23: mol_type = other RNA
caggtctgtgatgcccttagatg

SEQ ID NO: 192 - RNA, 35 nt, synthetic construct
  source 1..35: mol_type = other RNA
tacctggttgattctgccagtagtcatatgcttgt

SEQ ID NO: 193 - RNA, 24 nt, synthetic construct
  source 1..24: mol_type = other RNA
gttgggacccgaaagatggtgaac

SEQ ID NO: 194 - RNA, 30 nt, synthetic construct
  source 1..30: mol_type = other RNA
gaaaagcctctagttgatagaacaatgtag

SEQ ID NO: 195 - RNA, 18 nt, synthetic construct
  source 1..18: mol_type = other RNA
cgtggagcctgcggctta

SEQ ID NO: 196 - RNA, 39 nt, synthetic construct
  source 1..39: mol_type = other RNA
acttagaactggtacggacaaggggaatctgactgtcta

SEQ ID NO: 197 - RNA, 29 nt, synthetic construct
  source 1..29: mol_type = other RNA
tagtacgagaggaaccgttgattcagata

SEQ ID NO: 198 - RNA, 32 nt, synthetic construct
  source 1..32: mol_type = other RNA
ggattctccacggcaacgtaactgaacgcgga

SEQ ID NO: 199 - RNA, 27 nt, synthetic construct
  source 1..27: mol_type = other RNA
gaaaagcctctagttgatagaacaatg

SEQ ID NO: 200 - RNA, 22 nt, synthetic construct
  source 1..22: mol_type = other RNA
gaaaaggattggctctaagggt

SEQ ID NO: 201 - RNA, 29 nt, synthetic construct
  source 1..29: mol_type = other RNA
attccggggggcatgcctgttcgagcgtc

SEQ ID NO: 202 - RNA, 30 nt, synthetic construct
  source 1..30: mol_type = other RNA
cgccaaggatgttttcattaatcagtgaac

SEQ ID NO: 203 - RNA, 33 nt, synthetic construct
  source 1..33: mol_type = other RNA
tttgacctcggatcaggtaggagtacccgctga

SEQ ID NO: 204 - RNA, 22 nt, synthetic construct
  source 1..22: mol_type = other RNA
atagcgacgttgctttttgatc

SEQ ID NO: 205 - RNA, 27 nt, synthetic construct
  source 1..27: mol_type = other RNA
caaatgagaactttgaggactgaagtg

SEQ ID NO: 206 - RNA, 27 nt, synthetic construct
  source 1..27: mol_type = other RNA
caacttagaactggtacggacaagggg

SEQ ID NO: 207 - RNA, 27 nt, synthetic construct
  source 1..27: mol_type = other RNA
gaaagaagaccctgttgagcttgactc

SEQ ID NO: 208 - RNA, 27 nt, synthetic construct
  source 1..27: mol_type = other RNA
aagcgttggattgttcacccactaata

SEQ ID NO: 209 - RNA, 23 nt, synthetic construct
  source 1..23: mol_type = other RNA
tagaactggtacggacaagggga

SEQ ID NO: 210 - RNA, 33 nt, synthetic construct
  source 1..33: mol_type = other RNA
ggtttgacctcggatcaggtaggagtacccgct

SEQ ID NO: 211 - RNA, 31 nt, synthetic construct
  source 1..31: mol_type = other RNA
aaaaagctcgtagttgaaccttgggtctggc

SEQ ID NO: 212 - RNA, 23 nt, synthetic construct
  source 1..23: mol_type = other RNA
gaccgatagcgcacaagtagagt

SEQ ID NO: 213 - RNA, 31 nt, synthetic construct
  source 1..31: mol_type = other RNA
tgttgggacccgaaagatggtgaactatgcc

SEQ ID NO: 214 - RNA, 28 nt, synthetic construct
  source 1..28: mol_type = other RNA
ccaacttagaactggtacggacaagggg

SEQ ID NO: 215 - RNA, 36 nt, synthetic construct
  source 1..36: mol_type = other RNA
atttgtacggtgaaactgcgaatggctcattaaatc

SEQ ID NO: 216 - RNA, 38 nt, synthetic construct
  source 1..38: mol_type = other RNA
gcaaggctgaaacttaaagaaattgacggaagggcacc

SEQ ID NO: 217 - RNA, 29 nt, synthetic construct
  source 1..29: mol_type = other RNA
cgaccaacttagaactggtacggacaagg

SEQ ID NO: 218 - RNA, 24 nt, synthetic construct
  source 1..24: mol_type = other RNA
caaatcgatcgtcaaatttgggta

SEQ ID NO: 219 - RNA, 24 nt, synthetic construct
  source 1..24: mol_type = other RNA
acaataaatactgatacagggctc

SEQ ID NO: 220 - RNA, 19 nt, synthetic construct
  source 1..19: mol_type = other RNA
tcgcagcggttctgacgtg

SEQ ID NO: 221 - RNA, 23 nt, synthetic construct
  source 1..23: mol_type = other RNA
tagcgacgttgctttttgatcct

SEQ ID NO: 222 - RNA, 23 nt, synthetic construct
  source 1..23: mol_type = other RNA
tgcagatcttggtggtagtagca

SEQ ID NO: 223 - RNA, 26 nt, synthetic construct
  source 1..26: mol_type = other RNA
attgacagattgagagctctttcttg

SEQ ID NO: 224 - RNA, 33 nt, synthetic construct
  source 1..33: mol_type = other RNA
gcatatcaataagcggaggaaaagaaaccaaca

SEQ ID NO: 225 - RNA, 19 nt, synthetic construct
  source 1..19: mol_type = other RNA
atagggaacgtgagctggg

SEQ ID NO: 226 - RNA, 21 nt, synthetic construct
  source 1..21: mol_type = other RNA
caatgtagataagggaagtcg

SEQ ID NO: 227 - RNA, 43 nt, synthetic construct
  source 1..43: mol_type = other RNA
tttgacctcggatcaggtaggagtacccgctgaacttaagcat

SEQ ID NO: 228 - RNA, 34 nt, synthetic construct
  source 1..34: mol_type = other RNA
gtgcaaatcgatcgtcaaatttgggtataggggc

SEQ ID NO: 229 - RNA, 21 nt, synthetic construct
  source 1..21: mol_type = other RNA
tgttgacgcaatgtgatttct

SEQ ID NO: 230 - RNA, 37 nt, synthetic construct
  source 1..37: mol_type = other RNA
gaactttgaggactgaagtggggaaaggttccatgtg

SEQ ID NO: 231 - RNA, 34 nt, synthetic construct
  source 1..34: mol_type = other RNA
agcatatcaataagcggaggaaaagaaaccaaca

SEQ ID NO: 232 - RNA, 22 nt, synthetic construct
  source 1..22: mol_type = other RNA
agaactggtacggacaagggga

SEQ ID NO: 233 - RNA, 23 nt, synthetic construct
  source 1..23: mol_type = other RNA
atggaagtcggaatccgctaagg

SEQ ID NO: 234 - RNA, 21 nt, synthetic construct
  source 1..21: mol_type = other RNA
gcagatcttggtggtagtagc

SEQ ID NO: 235 - RNA, 24 nt, synthetic construct
  source 1..24: mol_type = other RNA
ccggcatcgatgaagaacgcagcg

SEQ ID NO: 236 - RNA, 22 nt, synthetic construct
  source 1..22: mol_type = other RNA
ttcgatgtcggctcttcctatc

SEQ ID NO: 237 - RNA, 22 nt, synthetic construct
  source 1..22: mol_type = other RNA
aaactttcaacaacggatctct

SEQ ID NO: 238 - RNA, 21 nt, synthetic construct
  source 1..21: mol_type = other RNA
agcggttctgacgtgcaaatc

SEQ ID NO: 239 - RNA, 21 nt, synthetic construct
  source 1..21: mol_type = other RNA
tgaaacttaaagaaattgacg

SEQ ID NO: 240 - RNA, 25 nt, synthetic construct
  source 1..25: mol_type = other RNA
tccggcatcgatgaagaacgcagcg

SEQ ID NO: 241 - RNA, 24 nt, synthetic construct
  source 1..24: mol_type = other RNA
aaaactttcaacaacggatctctt

SEQ ID NO: 242 - RNA, 21 nt, synthetic construct
  source 1..21: mol_type = other RNA
agaactggtacggacaagggg

SEQ ID NO: 243 - RNA, 26 nt, synthetic construct
  source 1..26: mol_type = other RNA
tgacaataaatactgatacagggctc

SEQ ID NO: 244 - RNA, 34 nt, synthetic construct
  source 1..34: mol_type = other RNA
tctagttgatagaacaatgtagataagggaagtc

SEQ ID NO: 245 - RNA, 30 nt, synthetic construct
  source 1..30: mol_type = other RNA
aatcgatcgtcaaatttgggtataggggcg

SEQ ID NO: 246 - RNA, 26 nt, synthetic construct
  source 1..26: mol_type = other RNA
agtggtgttgacgcaatgtgatttct

SEQ ID NO: 247 - RNA, 29 nt, synthetic construct
  source 1..29: mol_type = other RNA
tgacgtgcaaatcgatcgtcaaatttggg

SEQ ID NO: 248 - RNA, 33 nt, synthetic construct
  source 1..33: mol_type = other RNA
gttgatagaacaatgtagataagggaagtcggc

SEQ ID NO: 249 - RNA, 27 nt, synthetic construct
  source 1..27: mol_type = other RNA
gatagaacaatgtagataagggaagtc

SEQ ID NO: 250 - RNA, 48 nt, synthetic construct
  source 1..48: mol_type = other RNA
tttgacctcggatcaggtaggagtacccgctgaacttaagcatatcaa

SEQ ID NO: 251 - RNA, 37 nt, synthetic construct
  source 1..37: mol_type = other RNA
tctccaaggtgaaaagcctctagttgatagaacaatg

SEQ ID NO: 252 - RNA, 31 nt, synthetic construct
  source 1..31: mol_type = other RNA
agaactggtacggacaaggggaatctgactg

SEQ ID NO: 253 - RNA, 26 nt, synthetic construct
  source 1..26: mol_type = other RNA
aagtcggaatccgctaaggagtgtgt

SEQ ID NO: 254 - RNA, 25 nt, synthetic construct
  source 1..25: mol_type = other RNA
atcttggtggtagtagcaaatactc

SEQ ID NO: 255 - RNA, 29 nt, synthetic construct
  source 1..29: mol_type = other RNA
aatggatcgttactagtgggccatttttg

SEQ ID NO: 256 - RNA, 28 nt, synthetic construct
  source 1..28: mol_type = other RNA
gatgcgggatgaaccgaacgcgaggtta

SEQ ID NO: 257 - RNA, 31 nt, synthetic construct
  source 1..31: mol_type = other RNA
tgacgtgcaaatcgatcgtcaaatttgggta

SEQ ID NO: 258 - RNA, 29 nt, synthetic construct
  source 1..29: mol_type = other RNA
gtttgacctcggatcaggtaggagtaccc

SEQ ID NO: 259 - RNA, 30 nt, synthetic construct
  source 1..30: mol_type = other RNA
gtggctgttgggacccgaaagatggtgaac

SEQ ID NO: 260 - RNA, 24 nt, synthetic construct
  source 1..24: mol_type = other RNA
ttgttggtttctaggaccgccgta

SEQ ID NO: 261 - RNA, 27 nt, synthetic construct
  source 1..27: mol_type = other RNA
aaagaagaccctgttgagcttgactct

SEQ ID NO: 262 - RNA, 31 nt, synthetic construct
  source 1..31: mol_type = other RNA
gtttgacctcggatcaggtaggagtacccgc

SEQ ID NO: 263 - RNA, 37 nt, synthetic construct
  source 1..37: mol_type = other RNA
gaactggtacggacaaggggaatctgactgtctaatt

SEQ ID NO: 264 - RNA, 27 nt, synthetic construct
  source 1..27: mol_type = other RNA
cggattatggctgaacgcctctaagtc

SEQ ID NO: 265 - RNA, 26 nt, synthetic construct
  source 1..26: mol_type = other RNA
ccaacttagaactggtacggacaagg

SEQ ID NO: 266 - RNA, 30 nt, synthetic construct
  source 1..30: mol_type = other RNA
tcaaatgagaactttgaggactgaagtggg

SEQ ID NO: 267 - RNA, 19 nt, synthetic construct
  source 1..19: mol_type = other RNA
ttggtcatttagaggaagt

SEQ ID NO: 268 - RNA, 38 nt, synthetic construct
  source 1..38: mol_type = other RNA
tttgacctcggatcaggtaggagtacccgctgaactta

SEQ ID NO: 269 - RNA, 42 nt, synthetic construct
  source 1..42: mol_type = other RNA
gtttgacctcggatcaggtaggagtacccgctgaacttaagc

SEQ ID NO: 270 - RNA, 32 nt, synthetic construct
  source 1..32: mol_type = other RNA
tccgtgaatcatcgaatctttgaacgcacatt

SEQ ID NO: 271 - RNA, 34 nt, synthetic construct
  source 1..34: mol_type = other RNA
gacgttgctttttgatccttcgatgtcggctctt

SEQ ID NO: 272 - RNA, 32 nt, synthetic construct
  source 1..32: mol_type = other RNA
tagaactggtacggacaaggggaatctgactg

SEQ ID NO: 273 - RNA, 19 nt, synthetic construct
  source 1..19: mol_type = other RNA
aacgaacgagaccttaacc

SEQ ID NO: 274 - RNA, 23 nt, synthetic construct
  source 1..23: mol_type = other RNA
gaagaccctgttgagcttgactc

SEQ ID NO: 275 - RNA, 30 nt, synthetic construct
  source 1..30: mol_type = other RNA
tattccggggggcatgcctgttcgagcgtc

SEQ ID NO: 276 - RNA, 29 nt, synthetic construct
  source 1..29: mol_type = other RNA
aaagctcgtagttgaaccttgggtctggc

SEQ ID NO: 277 - RNA, 28 nt, synthetic construct
  source 1..28: mol_type = other RNA
tttttgatccttcgatgtcggctcttcc

SEQ ID NO: 278 - RNA, 46 nt, synthetic construct
  source 1..46: mol_type = other RNA
tttgacctcggatcaggtaggagtacccgctgaacttaagcatatc

(19) United States
(12) Patent Application Publication
(10) Pub. No.: US 2025/0215512 A1
(43) Pub. Date: Jul. 3, 2025

(54) METHODS OF TREATING DIMORPHIC FUNGAL DISEASES

(71) Applicant: REALSEQ BIOSCIENCES, INC., Santa Cruz, CA (US)

(72) Inventor: Sergio BARBERAN-SOLER, Santa Cruz, CA (US)

(21) Appl. No.: 18/853,311

(22) PCT Filed: Apr. 6, 2023
(86) PCT No.: PCT/US2023/017726

§ 371 Date: Oct. 1, 2024

Related U.S. Application Data
(60) Provisional application No. 63/328,363, filed on Apr. 7, 2022.

(51) Int. Cl. C12Q 1/6895; A61K 31/4196; C12Q 1/6813; C12Q 1/6855; C12Q 1/686; C12Q 1/6869

(52) U.S. Cl. CPC C12Q 1/6895; A61K 31/4196; C12Q 1/6813; C12Q 1/6855; C12Q 1/686; C12Q 1/6869

(57) ABSTRACT

Provided herein are methods for treating and diagnosing dimorphic fungal disorders.

### CROSS-REFERENCE

This application claims the benefit of U.S. Provisional Application No. 63/328,363, filed Apr. 7, 2022, which application is incorporated herein by reference.

### INCORPORATION BY REFERENCE OF SEQUENCE LISTING

The present application is being filed along with a Sequence Listing in electronic format. The Sequence Listing is provided as a file entitled 57767-713601.xml, created Apr. 3, 2023, which is 241 kilobytes in size. The information in the electronic format of the Sequence Listing is incorporated by reference in its entirety.

### SUMMARY

Dimorphic fungal diseases, including Valley Fever, are capable of causing severe illnesses. However, the current methods used to diagnose dimorphic fungal diseases are prone to false positives and false negatives. Additionally, current methods do not provide a way to differentiate between the acute and disseminating stages of disease. Providing an earlier or more accurate diagnosis may allow for more effective treatment. Provided herein are methods for predicting and diagnosing dimorphic fungal infections. Also provided herein are methods for detecting or predicting disseminating dimorphic infections.

In certain aspects, described herein is a method of detecting a dimorphic fungal infection in a subject comprising detecting at least one small RNA molecule in a sample from the subject associated with the dimorphic fungal infection. In some embodiments, the at least one small RNA molecule comprises at least 80% identity to one of SEQ ID NO: 1-120. In some embodiments, the at least one small RNA molecule is not a ribosomal RNA. In some embodiments, the dimorphic fungal infection is selected from the list consisting of coccidioidomycosis, histoplasmosis, blastomycosis, candidiasis, and a combination thereof. In some embodiments, the dimorphic fungal infection comprises coccidioidomycosis. In some embodiments, the dimorphic fungal infection comprises histoplasmosis. In some embodiments, the dimorphic fungal infection comprises blastomycosis. In some embodiments, the dimorphic fungal infection comprises candidiasis. In some embodiments, the dimorphic fungal infection results from an infection with a dimorphic fungal organism. In some embodiments, the dimorphic fungal organism is selected from the list consisting of Coccidioides, Histoplasma capsulatum, Blastomyces, Candida, Lomentospora prolificans, and Scedosporum. In some embodiments, the dimorphic fungal organism comprises a Coccidioides spp. In some embodiments, the dimorphic fungal organism comprises Histoplasma capsulatum. In some embodiments, the dimorphic fungal organism comprises a Blastomyces spp. In some embodiments, the dimorphic fungal organism comprises a Candida spp. In some embodiments, the at least one small RNA molecule is derived from the dimorphic fungal organism. In some embodiments, the at least one small RNA molecule is no more than 40 nucleotides in size. In some embodiments, detecting the at least one small RNA molecule comprises ligating a single-stranded adapter to the at least one small RNA molecule to produce a adapter-RNA molecule ligation product, wherein the polynucleotides of the plurality of adapter-polynucleotide ligation products comprise a 5′-proximal segment and a 3′-proximal segment, and wherein at least one of the 5′ proximal segment or 3′ proximal segment comprises a sequencing adapter; wherein the adapter comprises: a 5′-proximal segment and a 3′-proximal segment, wherein each proximal segment comprises at least one sequencing adapter, detection sequence, or a combination thereof, 5′-end and 3′-end groups that allow first and second consecutive ligation reactions either directly or after conversion of one or both of these end groups to ligatable 5′-end and 3′-end group(s); and, a template-deficient segment that restricts primer extension by a polymerase over the template-deficient segment. In some embodiments, detecting the at least one small RNA molecule further comprises circularizing the plurality of adapter-polynucleotide ligation products by ligating their 5′ ends to their 3′ ends to produce a plurality of circularized adapter-polynucleotide ligation products. In some embodiments, detecting the at least one small RNA molecule further comprises hybridizing a first primer comprising a sequence at least partially complementary to the 5′-proximal segment of the adapter, to the circularized adapter-polynucleotide ligation products. In some embodiments, detecting the at least one small RNA molecule further comprises extending the first primer with a polymerase to produce a plurality of monomeric nucleic acids, wherein each of the monomeric nucleic acids is complementary to: at least one sample polynucleotide of the plurality of sample polynucleotides flanked by at least a portion of the sequencing adapter. In some embodiments, detecting the at least one small RNA molecule further comprises amplifying the plurality of monomeric nucleic acids using the first primer and a second primer, wherein the sequence of the second primer is at least partially complementary to the 3′-proximal segment of the adapter, to produce amplicon(s) comprising the sequencing library. In some embodiments, the sample is selected from the list consisting of plasma, saliva, serum, and urine. In some embodiments, the sample comprises plasma. In some embodiments, the sample comprises saliva. In some embodiments, the sample comprises serum. In some embodiments, the sample comprises urine.

In certain aspects, described herein is a method of detecting a dimorphic fungal infection in a subject comprising: detecting at least one nucleic acid comprising any one of SEQ ID NO 1-120 in a sample from a subject. In some embodiments, the methods comprise determining that the subject is afflicted with the dimorphic fungal infection based on the presence of the at least one nucleic acid. In some embodiments, the methods comprise determining that the subject is afflicted with a chronic disease associated with the dimorphic fungal infection based on the presence of the at least one nucleic acid. In some embodiments, the methods comprise treating the subject with an antifungal. In some embodiments, the antifungal is selected from the list consisting of a amphotericin B compound, an azole, and a combination thereof. In some embodiments, the azole comprises Fluconazole. In some embodiments, detecting the at least one nucleic acid comprises obtaining the sample from a subject. In some embodiments, detecting the at least one nucleic acid comprises isolating at least one nucleic acid from the sample. In some embodiments, detecting the at least one nucleic acid comprises ligating an adapter to the at least one nucleic acid. In some embodiments, detecting the at least one nucleic acid further comprises depleting a second nucleic acid from the sample. In some embodiments, depleting a second nucleic acid from the sample comprises inhibiting the ligation of a second nucleic acid with an adapter. In some embodiments, the adapter comprises: a 5′-proximal segment and a 3′-proximal segment, wherein each proximal segment comprises at least one sequencing adapter, detection sequence, or a combination thereof; 5′-end and 3′-end groups that allow first and second consecutive ligation reactions either directly or after conversion of one or both of these end groups to ligatable 5′-end and 3′-end group(s); and, a template-deficient segment that restricts primer extension by a polymerase over said template-deficient segment. In some embodiments, the second nucleic acid comprises at least one sequence selected from SEQ ID NOs 121-278. In some embodiments, the methods comprise detecting at least two nucleic acids comprising any one of SEQ ID NO 1-120 in the sample from the subject. In some embodiments, the methods comprise detecting at least three amino acids comprising any one of SEQ ID NO 1-120 in the sample from the subject. In some embodiments, the dimorphic fungal disorder is selected from the list consisting of coccidioidomycosis, histoplasmosis, blastomycosis, candidiasis, and a combination thereof. In some embodiments, the dimorphic fungal disorder comprises coccidioidomycosis. In some embodiments, the dimorphic fungal infection results from an infection with a dimorphic fungal organism. In some embodiments, the dimorphic fungal organism is selected from the list consisting of Coccidioides, Histoplasma capsulatum, Blastomyces, Candida, Lomentospora prolificans, and Scedosporum. In some embodiments, the dimorphic fungal organism comprises a Coccidioides spp. In some embodiments, the at least one nucleic acid is derived from a dimorphic fungal organism causing the dimorphic fungal infection. In some embodiments, the at least one nucleic acid comprises an RNA. In some embodiments, the RNA is no more than 100, 90, 80, 70, 60, 50, 40, 30, 20, or 10 nucleotides in size. In some embodiments, the RNA is no more than 40 nucleotides in size. In some embodiments, the sample is selected from the list consisting of plasma, saliva, serum, and urine. In some embodiments, the at least one nucleic acid is detected by a nucleic acid amplification reaction. In some embodiments, the at least one nucleic acid is detected by qPCR. In some embodiments, the at least one nucleic acid is detected by next generation sequencing.

In certain aspects, described herein is a method of for preparing a sequencing library for a plurality of sample polynucleotides in a sample from a subject suspected of having a dimorphic fungal disorder, the method comprising: ligating a single-stranded adapter in the form of a plurality of nucleic acid residues to the plurality of sample polynucleotides to produce a plurality of adapter-polynucleotide ligation products, wherein the polynucleotides of the plurality of adapter-polynucleotide ligation products comprise a 5′-proximal segment and a 3′-proximal segment, and wherein at least one of the 5′ proximal segment or 3′ proximal segment comprises a sequencing adapter; wherein the adapter comprises: a 5′-proximal segment and a 3′-proximal segment, wherein each proximal segment comprises at least one sequencing adapter, detection sequence, or a combination thereof; 5′-end and 3′-end groups that allow first and second consecutive ligation reactions either directly or after conversion of one or both of these end groups to ligatable 5′-end and 3′-end group(s); and, a template-deficient segment that restricts primer extension by a polymerase over the template-deficient segment; circularizing the plurality of adapter-polynucleotide ligation products by ligating their 5′ ends to their 3′ ends to produce a plurality of circularized adapter-polynucleotide ligation products; hybridizing a first primer comprising a sequence at least partially complementary to the 5′-proximal segment of the adapter, to the circularized adapter-polynucleotide ligation products; extending the first primer with a polymerase to produce a plurality of monomeric nucleic acids, wherein each of the monomeric nucleic acids is complementary to: at least one sample polynucleotide of the plurality of sample polynucleotides flanked by at least a portion of the sequencing adapter; and amplifying the plurality of monomeric nucleic acids using the first primer and a second primer, wherein the sequence of the second primer is at least partially complementary to the 3′-proximal segment of the adapter, to produce amplicon(s) comprising the sequencing library. In some embodiments, at least one of plurality of nucleic acids comprises at least 80% identity to one of SEQ ID NO: 1-120. In some embodiments, the dimorphic fungal disorder is selected from the list consisting of coccidioidomycosis, histoplasmosis, blastomycosis, candidiasis, and a combination thereof. In some embodiments, the dimorphic fungal disorder comprises coccidioidomycosis. In some embodiments, the dimorphic fungal disorder comprises histoplasmosis. In some embodiments, the dimorphic fungal disorder comprises blastomycosis. In some embodiments, the dimorphic fungal disorder comprises candidiasis. In some embodiments, the dimorphic fungal disorder results from an infection with a dimorphic fungal organism. In some embodiments, the dimorphic fungal organism is selected from the list consisting of Coccidioides, Histoplasma capsulatum, Blastomyces, Candida, Lomentospora prolificans, and Scedosporum. In some embodiments, the dimorphic fungal organism comprises a Coccidioides spp. In some embodiments, the at least one nucleic acid is derived from the dimorphic fungal organism. In some embodiments, the at least one nucleic acid comprises an RNA. In some embodiments, the RNA is no more than 40 nucleotides in size. In some embodiments, the sample is selected from the list consisting of plasma, saliva, serum, and urine.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts small RNAseq alignments to 22 Coccidioides ncRNAs. Reads obtained from small RNA sequencing (RealSeq-Biofluids) of serum samples from 66 samples including: 10 negative controls, 26 patients with localized infection and 30 patients with disseminated infection, were aligned to a database of 22 Coccidioides ncRNAs. While the average length of the ncRNAs is 90nt, most reads obtained are around 20-25 nucleotides.

FIG. 2 depicts a principal component analysis of the Ribomarker signature.

FIG. 3 depicts an example of a method of preparing a sample.

### DETAILED DESCRIPTION

Provided herein are methods for the diagnosis and treatment of subjects with a dimorphic fungal disease. The most common endemic mycoses in the United States are coccidioidomycosis (Valley Fever), histoplasmosis, and blastomycosis. These important regional fungal diseases are caused by dimorphic fungi that occupy a specific ecological niche, are generally acquired through inhalation and unlike most fungal pathogens, can cause disease in healthy individuals.

Valley Fever, caused by the Coccidioides species, is endemic in the Western United States as well as in parts of Mexico, Central and South America and is 8 times more common than it was just 20 years ago. Valley Fever infection is caused by inhaling spores which become airborne from disturbances in the soil. In the United States, Arizona and California account for majority of cases with the CDC reporting a 32% increase in infections in 2019 alone and a 123% rise since 2014. Global warming and climate change are thought to be important factors affecting the rapid increase in Valley Fever cases while the ongoing drought in the Western US has fueled the recent expansion of endemic regions to include Southern Washington, Utah, and Colorado.

Valley Fever is an important public health problem causing severe illness in some patients-40% of infected patients develop acute or chronic disease (typically chronic pneumonia) while 1-3% of patients develop disseminated coccidioidomycosis, the most serious form (affecting skin, bones, joints and the CNS) which requires lifetime treatment (Table 1). Due to its nature, Valley Fever tends to disproportionately affect agricultural/farm and construction workers and creates a heavy lifetime cost on some disadvantaged populations. Although Valley Fever is considered an Orphan Disease by the FDA, endemic regions in some years exceed this classification and experts agree that there are more than 150,00 infections per year in the US. Treatment cost burdens are heavy, for example, lifetime treatment costs for patients diagnosed in 2019 with chronic or disseminated disease were estimated at $1.4bn in CA and AZ alone.

TABLE 1

| Total Infections | 150,000 | % |
|---|---|---|
| Medical attention | 50,000 | 33.33 |
| Diagnosed | 10,000 | 6.67 |
| Chronic pulmonary | 3,000 | 2 |
| Disseminated | 1,000 | 0.67 |
| Deaths | 160 | 0.11 |

Further, during the past decade, hospitalization costs for Valley Fever totaled $2.2 billion in California and Valley Fever is one of the most common causes of pneumonia in highly endemic regions. It is estimated that 15-30% of community acquired pneumonias (CAP) are Valley Fever infections, although they are rarely tested. Testing for Valley Fever is severely limited, although experts agree the number of infections is near 150,000 per year in the US, the CDC reports only 18,000 cases in 2019, the majority go undetected. Additionally, delays in treatment are related to the delays inherent in the current clinical laboratory-based tests primarily, culture, microscopy and serology which take weeks to receive results. Unfortunately, all current Valley Fever diagnostic tests are only available through reference laboratories and require a clinic visit for a blood draw or difficult biofluid extraction such as bronchial fluid; thus, access is greatly limited for many at-risk patient populations such as agriculture and farm workers.

Treatment for Valley Fever comprises 2 antifungal agents and is generally reserved for patients identified as at high risk of developing severe or disseminated infection (approximately 1% of total diagnosed patients), yet there are no accurate ways to stratify patients. Although various diagnostic techniques exist for Valley Fever; serology the most commonly used, is time consuming, lacks sensitivity and specificity with biological false negatives seen in approximately 50% of patients with early stage disease. This is particularly concerning as the most serious form of disseminated disease is often an early clinical event. Additionally, as Coccidioides cultures are infectious, confirmation of infections by culturing poses a real biohazard risk to clinical laboratory workers. The enzyme-linked immunosorbent assay (EIA) is also used and recommended for screening patients with suspected coccidioidomycosis. Even though the EIA has a sensitivity of greater than 90%, it suffers from high false-positive rates often requiring a confirmatory method such as immunodiffusion (ID) or complement fixation (CF) which are laborious, costly and subject to interpretation but are often necessary in order to make a definitive diagnosis. Newer, PCR-based tests have not made much forward progress as they are also high complexity tests with a high false negative rate and not recommended as a stand-alone test in any given scenario. Currently PCR-based tests require patient samples such as bronchial fluid, which is difficult to obtain and PCR-based tests have largely been reserved for difficult cases.

Early diagnosis of Valley Fever, as well as the ability to distinguish between acute and disseminated cases of Valley Fever, is critical as it leads to timely treatment, which may lessen the duration and severity of illness. Conversely, misdiagnosis and delayed treatment of Valley Fever can have devastating effects, including lifelong debilitation and even death. There is an unmet need for a rapid, sensitive test utilizing an easily obtainable biofluid such as saliva or blood to improve testing access and support earlier diagnosis. A point of need test would dramatically impact Valley Fever testing frequency in the relevant (including disadvantaged) populations allowing the disease to be identified at the earliest stages improving treatment options, potentially reducing misuse of antibiotic treatment of unidentified CAPs, and minimizing new infections by directing mitigation efforts such as mask wearing in agricultural fields or at particular job sites, for example.

### I. Methods

### A. Methods of Detection

Methods disclosed herein comprise detecting at least one nucleic acid from a subject. The methods comprise analyzing the nucleic acids in the sample to detect at least one of a presence, an absence, or a quantity of a nucleic acid sequence encompassing the nucleic acid of interest. In some embodiments, the methods comprise detecting at least two nucleic acids described herein. In some embodiments, the methods comprise detecting at least three nucleic acids described herein. In some embodiments, the methods comprise detecting at least four, five, six, seven, eight, nine, ten or more of the nucleic acids described herein. In some embodiments, the methods comprise detecting at least 20, 30, 40, 50, 60, 70, 80, 90, 100, 110, or 120 of the nucleic acids described herein.

In some cases, the nucleic acid sequence comprises DNA. In some instances, the nucleic acid sequence comprises a denatured DNA molecule or fragment thereof. In some instances, the nucleic acid sequence comprises DNA selected from: genomic DNA, viral DNA, mitochondrial DNA, plasmid DNA, amplified DNA, circular DNA, circulating DNA, cell-free DNA, or exosomal DNA. In some instances, the DNA is single-stranded DNA (ssDNA), double-stranded DNA, denaturing double-stranded DNA, synthetic DNA, and combinations thereof. The circular DNA may be cleaved or fragmented.

In some instances, the nucleic acid sequence comprises RNA. In some instances, the nucleic acid sequence comprises fragmented RNA. In some instances, the nucleic acid sequence comprises partially degraded RNA. In some instances, the nucleic acid comprises noncoding RNA (ncRNA). In some instances, the nucleic acid sequence comprises a microRNA or portion thereof. In some instances, the nucleic acid sequence comprises an RNA molecule or a fragmented RNA molecule (RNA fragments) selected from: a microRNA (miRNA), a pre-miRNA, a pri-miRNA, a mRNA, a pre-mRNA, circular RNA (circRNA), a ribosomal RNA (rRNA), a transfer RNA (tRNA), a pre-tRNA, a long non-coding RNA (lncRNA), a small nuclear RNA (snRNA), a circulating RNA, a cell-free RNA, an exosomal RNA, a vector-expressed RNA, an RNA transcript, a synthetic RNA, and combinations thereof. In some embodiments, the nucleic acid sequences is not a ribosomal RNA. In some embodiments, the nucleic acid sequence is not an 18s ribosomal RNA sequence.

In some embodiments, the nucleic acid is derived from the dimorphic fungal organism. In some embodiments, the nucleic acid derived from the dimorphic fungal organism comprises a nucleic acid encoding a protein produced by the dimorphic fungal organism, a nucleic acid from the genome of the dimorphic fungal organism, a nucleic acid transcribed from the genome of the dimorphic fungal organism, a nucleic acid complementary to a sequence of the genome of the dimorphic fungal organism, or a combination thereof.

In some embodiments, the nucleic acid is no more than about 100, 90, 80, 70, 60, 50, 40, 30, 20, or 10 nucleotides in size. In some embodiments, the nucleic acid is no more than about 100 nucleotides in size. In some embodiments, the nucleic acid is no more than about 90 nucleotides in size. In some embodiments, the nucleic acid is not more than about 90 nucleotides in size. In some embodiments, the nucleic acid is no more than about 80 nucleotides in size. In some embodiments, the nucleic acid is no more than about 70 nucleotides in size. In some embodiments, the nucleic acid is no more than about 60 nucleotides in size. In some embodiments, the nucleic acid is no more than about 50 nucleotides in size. In some embodiments, the nucleic acid is no more than about 40 nucleotides in size. In some embodiments, the nucleic acid is no more than about 30 nucleotides in size. In some embodiments, the nucleic acid is no more than about 20 nucleotides in size. In some embodiments, the nucleic acid is no more than about 10 nucleotides in size. In some embodiments, the nucleic acid is between 10 and 20 nucleotides, 10 and 30 nucleotides, 10 and 40 nucleotides, 10 and 50 nucleotides, 10 and 60 nucleotides, 10 and 70 nucleotides, 10 and 80 nucleotides, 10 and 90 nucleotides or 10 and 100 nucleotides.

In some embodiments, the RNA is no more than about 100, 90, 80, 70, 60, 50, 40, 30, 20, or 10 nucleotides in size. In some embodiments, the RNA is no more than about 100 nucleotides in size. In some embodiments, the RNA is no more than about 90 nucleotides in size. In some embodiments, the RNA is not more than about 90 nucleotides in size. In some embodiments, the RNA is no more than about 80 nucleotides in size. In some embodiments, the RNA is no more than about 70 nucleotides in size. In some embodiments, the RNA is no more than about 60 nucleotides in size. In some embodiments, the RNA is no more than about 50 nucleotides in size. In some embodiments, the RNA is no more than about 40 nucleotides in size. In some embodiments, the RNA is no more than about 30 nucleotides in size. In some embodiments, the RNA is no more than about 20 nucleotides in size. In some embodiments, the RNA is no more than about 10 nucleotides in size. In some embodiments, the RNA is between 10 and 20 nucleotides, 10 and 30 nucleotides, 10 and 40 nucleotides, 10 and 50 nucleotides, 10 and 60 nucleotides, 10 and 70 nucleotides, 10 and 80 nucleotides, 10 and 90 nucleotides or 10 and 100 nucleotides.

In some embodiments, the at least one nucleic acid comprises a nucleic acid comprising at least 80%, 85%, 90%, 95%, 97.5%, 98%, 99% or 100% sequence identity with one of SEQ ID NO: 1-120. In some embodiments, the at least one nucleic acid comprises a nucleic acid comprising at least 80%, 85%, 90%, 95%, 97.5%, 98%, 99% or 100% sequence identity with SEQ ID NO. 1. In some embodiments, the at least one nucleic acid comprises a nucleic acid comprising at least 80%, 85%, 90%, 95%, 97.5%, 98%, 99% or 100% sequence identity with SEQ ID NO. 2. In some embodiments, the at least one nucleic acid comprises a nucleic acid comprising at least 80%, 85%, 90%, 95%, 97.5%, 98%, 99% or 100% sequence identity with SEQ ID NO. 3. In some embodiments, the at least one nucleic acid comprises a nucleic acid comprising at least 80%, 85%, 90%, 95%, 97.5%, 98%, 99% or 100% sequence identity with SEQ ID NO. 4. In some embodiments, the at least one nucleic acid comprises a nucleic acid comprising at least 80%, 85%, 90%, 95%, 97.5%, 98%, 99% or 100% sequence identity with SEQ ID NO. 5. In some embodiments, the at least one nucleic acid comprises a nucleic acid comprising at least 80%, 85%, 90%, 95%, 97.5%, 98%, 99% or 100% sequence identity with SEQ ID NO. 6. In some embodiments, the at least one nucleic acid comprises a nucleic acid comprising at least 80%, 85%, 90%, 95%, 97.5%, 98%, 99% or 100% sequence identity with SEQ ID NO. 7. In some embodiments, the at least one nucleic acid comprises a nucleic acid comprising at least 80%, 85%, 90%, 95%, 97.5%, 98%, 99% or 100% sequence identity with SEQ ID NO. 8. In some embodiments, the at least one nucleic acid comprises a nucleic acid comprising at least 80%, 85%, 90%, 95%, 97.5%, 98%, 99% or 100% sequence identity with SEQ ID NO. 9. In some embodiments, the at least one nucleic acid comprises a nucleic acid comprising at least 80%, 85%, 90%, 95%, 97.5%, 98%, 99% or 100% sequence identity with SEQ ID NO. 10. In some embodiments, the at least one nucleic acid comprises a nucleic acid comprising at least 80%, 85%, 90%, 95%, 97.5%, 98%, 99% or 100% sequence identity with SEQ ID NO. 11. In some embodiments, the at least one nucleic acid comprises a nucleic acid comprising at least 80%, 85%, 90%, 95%, 97.5%, 98%, 99% or 100% sequence identity with SEQ ID NO. 12. In some embodiments, the at least one nucleic acid comprises a nucleic acid comprising at least 80%, 85%, 90%, 95%, 97.5%, 98%, 99% or 100% sequence identity with SEQ ID NO. 13. In some embodiments, the at least one nucleic acid comprises a nucleic acid comprising at least 80%, 85%, 90%, 95%, 97.5%, 98%, 99% or 100% sequence identity with SEQ ID NO. 14. In some embodiments, the at least one nucleic acid comprises a nucleic acid comprising at least 80%, 85%, 90%, 95%, 97.5%, 98%, 99% or 100% sequence identity with SEQ ID NO. 15. In some embodiments, the at least one nucleic acid comprises a nucleic acid comprising at least 80%, 85%, 90%, 95%, 97.5%, 98%, 99% or 100% sequence identity with SEQ ID NO. 16. In some embodiments, the at least one nucleic acid comprises a nucleic acid comprising at least 80%, 85%, 90%, 95%, 97.5%, 98%, 99% or 100% sequence identity with SEQ ID NO. 17. In some embodiments, the at least one nucleic acid comprises a nucleic acid comprising at least 80%, 85%, 90%, 95%, 97.5%, 98%, 99% or 100% sequence identity with SEQ ID NO. 18. In some embodiments, the at least one nucleic acid comprises a nucleic acid comprising at least 80%, 85%, 90%, 95%, 97.5%, 98%, 99% or 100% sequence identity with SEQ ID NO. 19. In some embodiments, the at least one nucleic acid comprises a nucleic acid comprising at least 80%, 85%, 90%, 95%, 97.5%, 98%, 99% or 100% sequence identity with SEQ ID NO. 20. In some embodiments, the at least one nucleic acid comprises a nucleic acid comprising at least 80%, 85%, 90%, 95%, 97.5%, 98%, 99% or 100% sequence identity with SEQ ID NO. 21. In some embodiments, the at least one nucleic acid comprises a nucleic acid comprising at least 80%, 85%, 90%, 95%, 97.5%, 98%, 99% or 100% sequence identity with SEQ ID NO. 22. In some embodiments, the at least one nucleic acid comprises a nucleic acid comprising at least 80%, 85%, 90%, 95%, 97.5%, 98%, 99% or 100% sequence identity with SEQ ID NO. 23. In some embodiments, the at least one nucleic acid comprises a nucleic acid comprising at least 80%, 85%, 90%, 95%, 97.5%, 98%, 99% or 100% sequence identity with SEQ ID NO. 24. In some embodiments, the at least one nucleic acid comprises a nucleic acid comprising at least 80%, 85%, 90%, 95%, 97.5%, 98%, 99% or 100% sequence identity with SEQ ID NO. 25. In some embodiments, the at least one nucleic acid comprises a nucleic acid comprising at least 80%, 85%, 90%, 95%, 97.5%, 98%, 99% or 100% sequence identity with SEQ ID NO. 26. In some embodiments, the at least one nucleic acid comprises a nucleic acid comprising at least 80%, 85%, 90%, 95%, 97.5%, 98%, 99% or 100% sequence identity with SEQ ID NO. 27. In some embodiments, the at least one nucleic acid comprises a nucleic acid comprising at least 80%, 85%, 90%, 95%, 97.5%, 98%, 99% or 100% sequence identity with SEQ ID NO. 28. In some embodiments, the at least one nucleic acid comprises a nucleic acid comprising at least 80%, 85%, 90%, 95%, 97.5%, 98%, 99% or 100% sequence identity with SEQ ID NO. 29. In some embodiments, the at least one nucleic acid comprises a nucleic acid comprising at least 80%, 85%, 90%, 95%, 97.5%, 98%, 99% or 100% sequence identity with SEQ ID NO. 30. In some embodiments, the at least one nucleic acid comprises a nucleic acid comprising at least 80%, 85%, 90%, 95%, 97.5%, 98%, 99% or 100% sequence identity with SEQ ID NO. 31. In some embodiments, the at least one nucleic acid comprises a nucleic acid comprising at least 80%, 85%, 90%, 95%, 97.5%, 98%, 99% or 100% sequence identity with SEQ ID NO. 32. In some embodiments, the at least one nucleic acid comprises a nucleic acid comprising at least 80%, 85%, 90%, 95%, 97.5%, 98%, 99% or 100% sequence identity with SEQ ID NO. 33. In some embodiments, the at least one nucleic acid comprises a nucleic acid comprising at least 80%, 85%, 90%, 95%, 97.5%, 98%, 99% or 100% sequence identity with SEQ ID NO. 34. In some embodiments, the at least one nucleic acid comprises a nucleic acid comprising at least 80%, 85%, 90%, 95%, 97.5%, 98%, 99% or 100% sequence identity with SEQ ID NO. 35. In some embodiments, the at least one nucleic acid comprises a nucleic acid comprising at least 80%, 85%, 90%, 95%, 97.5%, 98%, 99% or 100% sequence identity with SEQ ID NO. 36. In some embodiments, the at least one nucleic acid comprises a nucleic acid comprising at least 80%, 85%, 90%, 95%, 97.5%, 98%, 99% or 100% sequence identity with SEQ ID NO. 37. In some embodiments, the at least one nucleic acid comprises a nucleic acid comprising at least 80%, 85%, 90%, 95%, 97.5%, 98%, 99% or 100% sequence identity with SEQ ID NO. 38. In some embodiments, the at least one nucleic acid comprises a nucleic acid comprising at least 80%, 85%, 90%, 95%, 97.5%, 98%, 99% or 100% sequence identity with SEQ ID NO. 39. In some embodiments, the at least one nucleic acid comprises a nucleic acid comprising at least 80%, 85%, 90%, 95%, 97.5%, 98%, 99% or 100% sequence identity with SEQ ID NO. 40. In some embodiments, the at least one nucleic acid comprises a nucleic acid comprising at least 80%, 85%, 90%, 95%, 97.5%, 98%, 99% or 100% sequence identity with SEQ ID NO. 41. In some embodiments, the at least one nucleic acid comprises a nucleic acid comprising at least 80%, 85%, 90%, 95%, 97.5%, 98%, 99% or 100% sequence identity with SEQ ID NO. 42. In some embodiments, the at least one nucleic acid comprises a nucleic acid comprising at least 80%, 85%, 90%, 95%, 97.5%, 98%, 99% or 100% sequence identity with SEQ ID NO. 43. In some embodiments, the at least one nucleic acid comprises a nucleic acid comprising at least 80%, 85%, 90%, 95%, 97.5%, 98%, 99% or 100% sequence identity with SEQ ID NO. 44. In some embodiments, the at least one nucleic acid comprises a nucleic acid comprising at least 80%, 85%, 90%, 95%, 97.5%, 98%, 99% or 100% sequence identity with SEQ ID NO. 45. In some embodiments, the at least one nucleic acid comprises a nucleic acid comprising at least 80%, 85%, 90%, 95%, 97.5%, 98%, 99% or 100% sequence identity with SEQ ID NO. 46. In some embodiments, the at least one nucleic acid comprises a nucleic acid comprising at least 80%, 85%, 90%, 95%, 97.5%, 98%, 99% or 100% sequence identity with SEQ ID NO. 47. In some embodiments, the at least one nucleic acid comprises a nucleic acid comprising at least 80%, 85%, 90%, 95%, 97.5%, 98%, 99% or 100% sequence identity with SEQ ID NO. 48. In some embodiments, the at least one nucleic acid comprises a nucleic acid comprising at least 80%, 85%, 90%, 95%, 97.5%, 98%, 99% or 100% sequence identity with SEQ ID NO. 49. In some embodiments, the at least one nucleic acid comprises a nucleic acid comprising at least 80%, 85%, 90%, 95%, 97.5%, 98%, 99% or 100% sequence identity with SEQ ID NO. 50. In some embodiments, the at least one nucleic acid comprises a nucleic acid comprising at least 80%, 85%, 90%, 95%, 97.5%, 98%, 99% or 100% sequence identity with SEQ ID NO. 51. In some embodiments, the at least one nucleic acid comprises a nucleic acid comprising at least 80%, 85%, 90%, 95%, 97.5%, 98%, 99% or 100% sequence identity with SEQ ID NO. 52. In some embodiments, the at least one nucleic acid comprises a nucleic acid comprising at least 80%, 85%, 90%, 95%, 97.5%, 98%, 99% or 100% sequence identity with SEQ ID NO. 53. In some embodiments, the at least one nucleic acid comprises a nucleic acid comprising at least 80%, 85%, 90%, 95%, 97.5%, 98%, 99% or 100% sequence identity with SEQ ID NO. 54. In some embodiments, the at least one nucleic acid comprises a nucleic acid comprising at least 80%, 85%, 90%, 95%, 97.5%, 98%, 99% or 100% sequence identity with SEQ ID NO. 55. In some embodiments, the at least one nucleic acid comprises a nucleic acid comprising at least 80%, 85%, 90%, 95%, 97.5%, 98%, 99% or 100% sequence identity with SEQ ID NO. 56. In some embodiments, the at least one nucleic acid comprises a nucleic acid comprising at least 80%, 85%, 90%, 95%, 97.5%, 98%, 99% or 100% sequence identity with SEQ ID NO. 57. In some embodiments, the at least one nucleic acid comprises a nucleic acid comprising at least 80%, 85%, 90%, 95%, 97.5%, 98%, 99% or 100% sequence identity with SEQ ID NO. 58. In some embodiments, the at least one nucleic acid comprises a nucleic acid comprising at least 80%, 85%, 90%, 95%, 97.5%, 98%, 99% or 100% sequence identity with SEQ ID NO. 59. In some embodiments, the at least one nucleic acid comprises a nucleic acid comprising at least 80%, 85%, 90%, 95%, 97.5%, 98%, 99% or 100% sequence identity with SEQ ID NO. 60. In some embodiments, the at least one nucleic acid comprises a nucleic acid comprising at least 80%, 85%, 90%, 95%, 97.5%, 98%, 99% or 100% sequence identity with SEQ ID NO. 61. In some embodiments, the at least one nucleic acid comprises a nucleic acid comprising at least 80%, 85%, 90%, 95%, 97.5%, 98%, 99% or 100% sequence identity with SEQ ID NO. 62. In some embodiments, the at least one nucleic acid comprises a nucleic acid comprising at least 80%, 85%, 90%, 95%, 97.5%, 98%, 99% or 100% sequence identity with SEQ ID NO. 63. In some embodiments, the at least one nucleic acid comprises a nucleic acid comprising at least 80%, 85%, 90%, 95%, 97.5%, 98%, 99% or 100% sequence identity with SEQ ID NO. 64. In some embodiments, the at least one nucleic acid comprises a nucleic acid comprising at least 80%, 85%, 90%, 95%, 97.5%, 98%, 99% or 100% sequence identity with SEQ ID NO. 65. In some embodiments, the at least one nucleic acid comprises a nucleic acid comprising at least 80%, 85%, 90%, 95%, 97.5%, 98%, 99% or 100% sequence identity with SEQ ID NO. 66. In some embodiments, the at least one nucleic acid comprises a nucleic acid comprising at least 80%, 85%, 90%, 95%, 97.5%, 98%, 99% or 100% sequence identity with SEQ ID NO. 67. In some embodiments, the at least one nucleic acid comprises a nucleic acid comprising at least 80%, 85%, 90%, 95%, 97.5%, 98%, 99% or 100% sequence identity with SEQ ID NO. 68. In some embodiments, the at least one nucleic acid comprises a nucleic acid comprising at least 80%, 85%, 90%, 95%, 97.5%, 98%, 99% or 100% sequence identity with SEQ ID NO. 69. In some embodiments, the at least one nucleic acid comprises a nucleic acid comprising at least 80%, 85%, 90%, 95%, 97.5%, 98%, 99% or 100% sequence identity with SEQ ID NO. 70. In some embodiments, the at least one nucleic acid comprises a nucleic acid comprising at least 80%, 85%, 90%, 95%, 97.5%, 98%, 99% or 100% sequence identity with SEQ ID NO. 71. In some embodiments, the at least one nucleic acid comprises a nucleic acid comprising at least 80%, 85%, 90%, 95%, 97.5%, 98%, 99% or 100% sequence identity with SEQ ID NO. 72. In some embodiments, the at least one nucleic acid comprises a nucleic acid comprising at least 80%, 85%, 90%, 95%, 97.5%, 98%, 99% or 100% sequence identity with SEQ ID NO. 73. In some embodiments, the at least one nucleic acid comprises a nucleic acid comprising at least 80%, 85%, 90%, 95%, 97.5%, 98%, 99% or 100% sequence identity with SEQ ID NO. 74. In some embodiments, the at least one nucleic acid comprises a nucleic acid comprising at least 80%, 85%, 90%, 95%, 97.5%, 98%, 99% or 100% sequence identity with SEQ ID NO. 75. In some embodiments, the at least one nucleic acid comprises a nucleic acid comprising at least 80%, 85%, 90%, 95%, 97.5%, 98%, 99% or 100% sequence identity with SEQ ID NO. 76. In some embodiments, the at least one nucleic acid comprises a nucleic acid comprising at least 80%, 85%, 90%, 95%, 97.5%, 98%, 99% or 100% sequence identity with SEQ ID NO. 77. In some embodiments, the at least one nucleic acid comprises a nucleic acid comprising at least 80%, 85%, 90%, 95%, 97.5%, 98%, 99% or 100% sequence identity with SEQ ID NO. 78. In some embodiments, the at least one nucleic acid comprises a nucleic acid comprising at least 80%, 85%, 90%, 95%, 97.5%, 98%, 99% or 100% sequence identity with SEQ ID NO. 79. In some embodiments, the at least one nucleic acid comprises a nucleic acid comprising at least 80%, 85%, 90%, 95%, 97.5%, 98%, 99% or 100% sequence identity with SEQ ID NO. 80. In some embodiments, the at least one nucleic acid comprises a nucleic acid comprising at least 80%, 85%, 90%, 95%, 97.5%, 98%, 99% or 100% sequence identity with SEQ ID NO. 81. In some embodiments, the at least one nucleic acid comprises a nucleic acid comprising at least 80%, 85%, 90%, 95%, 97.5%, 98%, 99% or 100% sequence identity with SEQ ID NO. 82. In some embodiments, the at least one nucleic acid comprises a nucleic acid comprising at least 80%, 85%, 90%, 95%, 97.5%, 98%, 99% or 100% sequence identity with SEQ ID NO. 83. In some embodiments, the at least one nucleic acid comprises a nucleic acid comprising at least 80%, 85%, 90%, 95%, 97.5%, 98%, 99% or 100% sequence identity with SEQ ID NO. 84. In some embodiments, the at least one nucleic acid comprises a nucleic acid comprising at least 80%, 85%, 90%, 95%, 97.5%, 98%, 99% or 100% sequence identity with SEQ ID NO. 85. In some embodiments, the at least one nucleic acid comprises a nucleic acid comprising at least 80%, 85%, 90%, 95%, 97.5%, 98%, 99% or 100% sequence identity with SEQ ID NO. 86. In some embodiments, the at least one nucleic acid comprises a nucleic acid comprising at least 80%, 85%, 90%, 95%, 97.5%, 98%, 99% or 100% sequence identity with SEQ ID NO. 87. In some embodiments, the at least one nucleic acid comprises a nucleic acid comprising at least 80%, 85%, 90%, 95%, 97.5%, 98%, 99% or 100% sequence identity with SEQ ID NO. 88. In some embodiments, the at least one nucleic acid comprises a nucleic acid comprising at least 80%, 85%, 90%, 95%, 97.5%, 98%, 99% or 100% sequence identity with SEQ ID NO. 89. In some embodiments, the at least one nucleic acid comprises a nucleic acid comprising at least 80%, 85%, 90%, 95%, 97.5%, 98%, 99% or 100% sequence identity with SEQ ID NO. 90. In some embodiments, the at least one nucleic acid comprises a nucleic acid comprising at least 80%, 85%, 90%, 95%, 97.5%, 98%, 99% or 100% sequence identity with SEQ ID NO. 91. In some embodiments, the at least one nucleic acid comprises a nucleic acid comprising at least 80%, 85%, 90%, 95%, 97.5%, 98%, 99% or 100% sequence identity with SEQ ID NO. 92. In some embodiments, the at least one nucleic acid comprises a nucleic acid comprising at least 80%, 85%, 90%, 95%, 97.5%, 98%, 99% or 100% sequence identity with SEQ ID NO. 93. In some embodiments, the at least one nucleic acid comprises a nucleic acid comprising at least 80%, 85%, 90%, 95%, 97.5%, 98%, 99% or 100% sequence identity with SEQ ID NO. 94. In some embodiments, the at least one nucleic acid comprises a nucleic acid comprising at least 80%, 85%, 90%, 95%, 97.5%, 98%, 99% or 100% sequence identity with SEQ ID NO. 95. In some embodiments, the at least one nucleic acid comprises a nucleic acid comprising at least 80%, 85%, 90%, 95%, 97.5%, 98%, 99% or 100% sequence identity with SEQ ID NO. 96. In some embodiments, the at least one nucleic acid comprises a nucleic acid comprising at least 80%, 85%, 90%, 95%, 97.5%, 98%, 99% or 100% sequence identity with SEQ ID NO. 97. In some embodiments, the at least one nucleic acid comprises a nucleic acid comprising at least 80%, 85%, 90%, 95%, 97.5%, 98%, 99% or 100% sequence identity with SEQ ID NO. 98. In some embodiments, the at least one nucleic acid comprises a nucleic acid comprising at least 80%, 85%, 90%, 95%, 97.5%, 98%, 99% or 100% sequence identity with SEQ ID NO. 99. In some embodiments, the at least one nucleic acid comprises a nucleic acid comprising at least 80%, 85%, 90%, 95%, 97.5%, 98%, 99% or 100% sequence identity with SEQ ID NO. 100. In some embodiments, the at least one nucleic acid comprises a nucleic acid comprising at least 80%, 85%, 90%, 95%, 97.5%, 98%, 99% or 100% sequence identity with SEQ ID NO. 101. In some embodiments, the at least one nucleic acid comprises a nucleic acid comprising at least 80%, 85%, 90%, 95%, 97.5%, 98%, 99% or 100% sequence identity with SEQ ID NO. 102. In some embodiments, the at least one nucleic acid comprises a nucleic acid comprising at least 80%, 85%, 90%, 95%, 97.5%, 98%, 99% or 100% sequence identity with SEQ ID NO. 103. In some embodiments, the at least one nucleic acid comprises a nucleic acid comprising at least 80%, 85%, 90%, 95%, 97.5%, 98%, 99% or 100% sequence identity with SEQ ID NO. 104. In some embodiments, the at least one nucleic acid comprises a nucleic acid comprising at least 80%, 85%, 90%, 95%, 97.5%, 98%, 99% or 100% sequence identity with SEQ ID NO. 105. In some embodiments, the at least one nucleic acid comprises a nucleic acid comprising at least 80%, 85%, 90%, 95%, 97.5%, 98%, 99% or 100% sequence identity with SEQ ID NO. 106. In some embodiments, the at least one nucleic acid comprises a nucleic acid comprising at least 80%, 85%, 90%, 95%, 97.5%, 98%, 99% or 100% sequence identity with SEQ ID NO. 107. In some embodiments, the at least one nucleic acid comprises a nucleic acid comprising at least 80%, 85%, 90%, 95%, 97.5%, 98%, 99% or 100% sequence identity with SEQ ID NO. 108. In some embodiments, the at least one nucleic acid comprises a nucleic acid comprising at least 80%, 85%, 90%, 95%, 97.5%, 98%, 99% or 100% sequence identity with SEQ ID NO. 109. In some embodiments, the at least one nucleic acid comprises a nucleic acid comprising at least 80%, 85%, 90%, 95%, 97.5%, 98%, 99% or 100% sequence identity with SEQ ID NO. 110. In some embodiments, the at least one nucleic acid comprises a nucleic acid comprising at least 80%, 85%, 90%, 95%, 97.5%, 98%, 99% or 100% sequence identity with SEQ ID NO. 111. In some embodiments, the at least one nucleic acid comprises a nucleic acid comprising at least 80%, 85%, 90%, 95%, 97.5%, 98%, 99% or 100% sequence identity with SEQ ID NO. 112. In some embodiments, the at least one nucleic acid comprises a nucleic acid comprising at least 80%, 85%, 90%, 95%, 97.5%, 98%, 99% or 100% sequence identity with SEQ ID NO. 113. In some embodiments, the at least one nucleic acid comprises a nucleic acid comprising at least 80%, 85%, 90%, 95%, 97.5%, 98%, 99% or 100% sequence identity with SEQ ID NO. 114. In some embodiments, the at least one nucleic acid comprises a nucleic acid comprising at least 80%, 85%, 90%, 95%, 97.5%, 98%, 99% or 100% sequence identity with SEQ ID NO. 115. In some embodiments, the at least one nucleic acid comprises a nucleic acid comprising at least 80%, 85%, 90%, 95%, 97.5%, 98%, 99% or 100% sequence identity with SEQ ID NO. 116. In some embodiments, the at least one nucleic acid comprises a nucleic acid comprising at least 80%, 85%, 90%, 95%, 97.5%, 98%, 99% or 100% sequence identity with SEQ ID NO. 117. In some embodiments, the at least one nucleic acid comprises a nucleic acid comprising at least 80%, 85%, 90%, 95%, 97.5%, 98%, 99% or 100% sequence identity with SEQ ID NO. 118. In some embodiments, the at least one nucleic acid comprises a nucleic acid comprising at least 80%, 85%, 90%, 95%, 97.5%, 98%, 99% or 100% sequence identity with SEQ ID NO. 119. In some embodiments, the at least one nucleic acid comprises a nucleic acid comprising at least 80%, 85%, 90%, 95%, 97.5%, 98%, 99% or 100% sequence identity with SEQ ID NO. 120.

Nucleic acid-based detection techniques that may be useful for the methods herein include quantitative polymerase chain reaction (qPCR), gel electrophoresis, immunochemistry, in situ hybridization such as fluorescent in situ hybridization (FISH), cytochemistry, and next generation sequencing. In some embodiments, the methods involve TaqMan™ qPCR, which involves a nucleic acid amplification reaction with a specific primer pair, and hybridization of the amplified nucleic acids with a hydrolysable probe specific to a target nucleic acid.

In some instances, the methods involve hybridization and/or amplification assays that include, but are not limited to, Southern or Northern analyses, polymerase chain reaction analyses, isothermal amplification, and probe arrays. Non-limiting amplification reactions include, but are not limited to, qPCR, self-sustained sequence replication, transcriptional amplification system, Q-Beta Replicase, rolling circle replication, or any other nucleic acid amplification known in the art. As discussed, reference to qPCR herein includes use of TaqMan™ methods. One example of a hybridization assay includes the use of nucleic acid probes conjugated or otherwise immobilized on a bead, multi-well plate, or other substrate, wherein the nucleic acid probes are configured to hybridize with a target nucleic acid sequence of a genotype provided herein. A non-limiting method is one employed in Anal Chem. 2013 Feb. 5; 85(3):1932-9.

In some embodiments, the nucleic acid is identified by amplification. In some embodiments, the nucleic acid is identified by next generation sequencing. In some embodiments, the nucleic acid is identified by qPCR. In some instances, primers and/or probes described herein for detecting a target nucleic acid are used in an amplification reaction. In some instances, the amplification reaction is qPCR. One example of a qPCR is a method employing a TaqMan™ assay. “Wt_Probe_Hex” and “Mut_Probe_FAM” mean “Wild type_probes_tagged with HEX reporter dye” and “Mut_probe_tagged with FAM reporter dye”, respectively. “+” stands for LNA bases (Locked nucleotides), which are analogues that are modified at 2′-O, 4′-C and form a bridge. This bridge results in restricted base pairing giving room to adjust the Tm as needed between the probes. Thus, +A, +T, +C or +G signify A, T, G or C bases are added on the modified backbone.

In some instances, qPCR comprises using an intercalating dye. Examples of intercalating dyes include SYBR green I, SYBR green II, SYBR gold, ethidium bromide, methylene blue, Pyronin Y, DAPI, acridine orange, Blue View or phycoerythrin. In some instances, the intercalating dye is SYBR.

In one aspect, the methods provided herein comprise an amplification reaction such as qPCR. In one embodiment, genetic material is obtained from a sample of a subject, e.g., a sample of blood or serum. In certain embodiments where nucleic acids are extracted, the nucleic acids are extracted using any technique that does not interfere with subsequent analysis. In certain embodiments, this technique uses alcohol precipitation using ethanol, methanol, or isopropyl alcohol. In certain embodiments, this technique uses phenol, chloroform, or any combination thereof. In certain embodiments, this technique uses cesium chloride. In certain embodiments, this technique uses sodium, potassium or ammonium acetate or any other salt commonly used to precipitate DNA. In certain embodiments, this technique utilizes a column or resin based nucleic acid purification scheme such as those commonly sold commercially, one non-limiting example would be the GenElute Bacterial Genomic DNA Kit available from Sigma Aldrich. In certain embodiments, after extraction the nucleic acid is stored in water, Tris buffer, or Tris-EDTA buffer before subsequent analysis. In some embodiments, the nucleic acid material is extracted in water. In some cases, extraction does not comprise nucleic acid purification.

In one embodiment of a qPCR assay, the nucleic acid sample is combined with primers and probes specific for a target nucleic acid that may or may not be present in the sample, and a DNA polymerase. An amplification reaction is performed with a thermal cycler that heats and cools the sample for nucleic acid amplification, and illuminates the sample at a specific wavelength to excite a fluorophore on the probe and detect the emitted fluorescence. For TaqMan™ methods, the probe may be a hydrolysable probe comprising a fluorophore and quencher that is hydrolyzed by DNA polymerase when hybridized to a target nucleic acid. In some cases, the presence of a target nucleic acid is determined when the number of amplification cycles to reach a threshold value is less than 30, 29, 28, 27, 26, 25, 24, 23, 22, 21, or 20 cycles.

In some embodiments, the methods described herein comprises sequencing genetic material from the subject. Sequencing can be performed with any appropriate sequencing technology, including but not limited to single-molecule real-time (SMRT) sequencing, Polony sequencing, sequencing by ligation, sequencing by synthesis, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis. Sequencing methods also include next-generation sequencing, e.g., modern sequencing technologies such as Illumina sequencing (e.g., Solexa), Oxford Nanopore sequencing, Roche 454 sequencing, Ion torrent sequencing, and SOLID sequencing. In some cases, next-generation sequencing involves high-throughput sequencing methods. Additional sequencing methods available to one of skill in the art may also be employed.

In some embodiments, detecting the at least one nucleic acid is predictive that the subject has a disseminating dimorphic fungal disease with a specificity of at least about 70%. In some embodiments, detecting the at least one nucleic acid is predictive that the subject has a disseminating dimorphic fungal disease with a specificity of at least about 75%. In some embodiments, detecting the at least one nucleic acid is predictive that the subject has a disseminating dimorphic fungal disease with a specificity of at least about 80%. In some embodiments, detecting the at least one nucleic acid is predictive that the subject has a disseminating dimorphic fungal disease with a specificity of at least about 85%. In some embodiments, detecting the at least one nucleic acid is predictive that the subject has a disseminating dimorphic fungal disease with a specificity of at least about 90%. In some embodiments, detecting the at least one nucleic acid is predictive that the subject has a disseminating dimorphic fungal disease with a specificity of at least about 95%.

### B. Methods of Preparing a Sample

In some embodiments, the methods comprise preparing a sample from a subject as described herein. In some embodiments, a nucleic acid is isolated from the sample. In some embodiments, the nucleic acid is detected directly from the sample. In some embodiments, the nucleic acid is isolated or purified from the sample. In some embodiments, the nucleic acid is RNA. In some embodiments, the nucleic acid is miRNA. In some embodiments, the methods comprise an RNA extraction step. In some embodiments, the methods do not comprise an RNA extraction step.

In some embodiments, the methods described herein comprise detecting a nucleic acid in a biological sample as described herein. In some embodiments, the biological sample comprises blood, serum, saliva, or plasma. In some embodiments, the biological sample comprises serum. In some embodiments, the biological sample comprises saliva. In some embodiments, the biological sample comprises plasma. In some embodiments, the biological sample comprises blood. In some embodiments, the biological sample comprises urine.

In some embodiments, an adapter is ligated to the nucleic acid to form a nucleic acid-adapter complex. In some embodiments, the adapter is ligated to the 3′ end of the nucleic acid. In some embodiments, the adapter is ligated to the 5′ end of the nucleic acid. In some embodiments, a blocking agent is added to the reaction. In some embodiment, the nucleic acid-adapter complex is circularized. In some embodiments, the methods comprise reverse transcribing the nucleic acid-adapter complex. In some embodiments, the methods comprise enzymatically amplifying the reverse transcript, or a portion thereof, to produce an amplified polynucleotide.

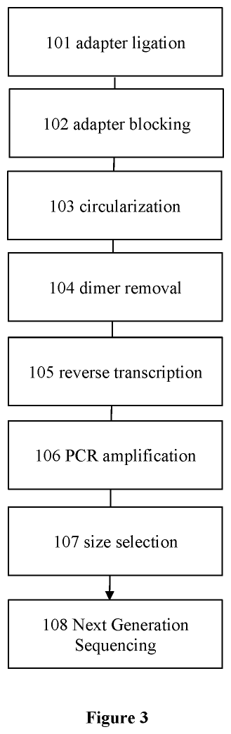

In some embodiments, the sample may be prepared by a method comprising at least one of the steps described in FIG. 3. An adapter may be ligated to the nucleic acid 101. A blocking agent may be used 102. The adapter-nucleic acid complex may be circularized 103. Adapter dimers may be removed from the solution 104. Reverse transcription of the nucleic acid may occur 105. The resulting circular DNA may be amplified using PCR 106. The resulting PCR amplicons may be selected by size 107. The purified nucleic acid molecules may be sequenced 108 (e.g. next generation sequencing).

In some embodiments, the method further comprises depleting a second nucleic acid from the sample. In some embodiments, the method comprises depleting a plurality of nucleic acids from the sample. In some embodiments, the second nucleic acid comprises at least one sequence selected from SEQ ID NOs: 121-278. In some embodiments, depleting a second nucleic acid from the sample comprises inhibiting the ligation of a second nucleic acid with an adapter. In some embodiments, at least one sequence selected from SEQ ID NOs 121-278 is removed from a dataset after sequencing has occurred.

In some embodiments, the method comprises blocking, removing, filtering, or disregarding at least one sequence selected from SEQ ID NOs 121-278. In some embodiments, the blocking, removing, filtering, or disregarding occurs before adapter ligation. In some embodiments, the blocking, removing, filtering, or disregarding occurs before adapter blocking. In some embodiments, the blocking, removing, filtering, or disregarding occurs before circularization. In some embodiments, the blocking, removing, filtering, or disregarding occurs before dimer removal. In some embodiments, the blocking, removing, filtering, or disregarding occurs before reverse transcription. In some embodiments, the blocking, removing, filtering, or disregarding occurs before PCT amplification. In some embodiments, the blocking, removing, filtering, or disregarding occurs before size selection. In some embodiments, the blocking, removing, filtering, or disregarding occurs before sequencing. In some embodiments, the blocking, removing, filtering, or disregarding occurs after sequencing.

In some embodiments, the adapter comprises an adapter. In some embodiments, the adapter is in the form of a single-stranded nucleic acid comprising: a) a single-stranded 5′-proximal segment and a single-stranded 3′-proximal segment, wherein the single-stranded 5′-proximal segment and the single-stranded 3′ proximal segment comprise a sequencing adapter, a detection sequence, or a combination thereof; b) 5′-end and 3′-end groups that allow first and second consecutive ligation reactions either directly or after conversion of one or both 5′-end and 3′-end groups to ligatable 5′-end and ligatable 3′-end group(s); and c) a template-deficient segment linking the single-stranded 5′ proximal segment and the single-stranded 3′ proximal segment that restricts primer extension by a polymerase over the template-deficient segment. In some embodiments, the adapter comprises at least one DNA residue and at least one RNA residue. In some embodiments, the adapter comprises at least one RNA residue, one DNA residue, modified nucleic acid residue, or non-nucleotide residue, or a combination thereof. In some embodiments, the adapter comprises at least one sequence selected from: a sequencing adapter, a primer binding site, a detection sequence, a probe hybridization sequence, a capture oligonucleotide binding site, a polymerase binding site, an endonuclease restriction site, a sequencing bar-code, an indexing sequence, a Zip-code, one or more random nucleotides, a unique molecular identifier (UMI), and a sequencing flow-cell binding site, and combinations thereof. In some embodiments, the 5′-end and/or 3′-end of the adapter contains a reversible blocking group preventing circularization or concatamerization of the adapter while allowing its ligation with a sample polynucleotide, wherein the reversible blocking group requires chemical, photochemical or enzymatic conversion to an active end group prior to the circularization of the adapter-polynucleotide ligation product. In some embodiments, the reversible blocking group is a 3′-end-blocking group selected from: 3′-p, 2′,3′-cyclic phosphate (2′,3′>p), 3′-O-(α-methoxyethyl) ether, and 3′-O-isovaleryl ester.

In some embodiments, the method comprises ligating a single-stranded adapter in the form of a plurality of nucleic acid residues to the plurality of sample polynucleotides to produce a plurality of adapter-polynucleotide ligation products, wherein the polynucleotides of the plurality of adapter-polynucleotide ligation products comprise a 5′-proximal segment and a 3′-proximal segment, and wherein at least one of the 5′ proximal segment or 3′ proximal segment comprises a sequencing adapter. In some embodiments, the method comprises circularizing the plurality of adapter-polynucleotide ligation products by ligating their 5′ ends to their 3′ ends to produce a plurality of circularized adapter-polynucleotide ligation products. In some embodiments, the method comprises hybridizing a first primer comprising a sequence at least partially complementary to the 5′-proximal segment of the adapter, to the circularized adapter-polynucleotide ligation products. In some embodiments, the method comprises extending the first primer with a polymerase to produce a plurality of monomeric nucleic acids, wherein each of the monomeric nucleic acids is complementary to: at least one sample polynucleotide of the plurality of sample polynucleotides flanked by at least a portion of the sequencing adapter. In some embodiments, the method comprises amplifying the plurality of monomeric nucleic acids using the first primer and a second primer, wherein the sequence of the second primer is at least partially complementary to the 3′-proximal segment of the adapter, to produce amplicon(s) comprising the sequencing library.

In some embodiments, disclosed herein is a method for preparing a sequencing library for a plurality of sample polynucleotides in a sample, wherein the subject is suspected of having a dimorphic fungal disorder. In some embodiments, the method comprises ligating a single-stranded adapter in the form of a plurality of nucleic acid residues to the plurality of sample polynucleotides to produce a plurality of adapter-polynucleotide ligation products, wherein the polynucleotides of the plurality of adapter-polynucleotide ligation products comprise a 5′-proximal segment and a 3′-proximal segment, and wherein at least one of the 5′ proximal segment or 3′ proximal segment comprises a sequencing adapter. In some embodiments, the adapter comprises a 5′-proximal segment and a 3′-proximal segment, wherein each proximal segment comprises at least one sequencing adapter, detection sequence, or a combination thereof, 5′-end and 3′-end groups that allow first and second consecutive ligation reactions either directly or after conversion of one or both of these end groups to ligatable 5′-end and 3′-end group(s); and, a template-deficient segment that restricts primer extension by a polymerase over the template-deficient segment. In some embodiments, the method comprises circularizing the plurality of adapter-polynucleotide ligation products by ligating their 5′ ends to their 3′ ends to produce a plurality of circularized adapter-polynucleotide ligation products. In some embodiments, the method comprises hybridizing a first primer comprising a sequence at least partially complementary to the 5′-proximal segment of the adapter, to the circularized adapter-polynucleotide ligation products. In some embodiments, the method comprises extending the first primer with a polymerase to produce a plurality of monomeric nucleic acids, wherein each of the monomeric nucleic acids is complementary to: at least one sample polynucleotide of the plurality of sample polynucleotides flanked by at least a portion of the sequencing adapter. In some embodiments, the method comprises amplifying the plurality of monomeric nucleic acids using the first primer and a second primer, wherein the sequence of the second primer is at least partially complementary to the 3′-proximal segment of the adapter, to produce amplicon(s) comprising the sequencing library.

In some embodiments, the method comprises circularizing the plurality of adapter-polynucleotide ligation products by ligating their 5′ ends to their 3′ ends to produce a plurality of circularized adapter-polynucleotide ligation products. In some embodiments, the ligating and/or circularizing comprise contacting the adapter and/or the plurality of adapter-polynucleotide ligation products with a ligase. The ligase may include, without limitations, 3′-OH ligase ligating 5′-p to 3′-OH or 5′-App to 3′-OH; 5′-OH ligase ligating 5′-p to 3-phosphorylated end selected from: 3′-p, 2′,3′>p or 2′-p; and combinations thereof.

The method may comprise converting the 5′-end and/or 3′-end groups of the adapter-polynucleotide ligation product before the circularizing. In some embodiments, converting comprises a conversion selected from: a) 5′-OH to 5′-p; b) 3′-p to 3′-OH; c) 2′-p to 2′-OH; and d) 2′,3′>p to 2′-OH/3′-OH, or combinations thereof.

The method may further comprise depleting, separating, degrading, and/or blocking a remaining unligated adapter after the ligating to the sample polynucleotide and/or after the circularizing. In some embodiments, blocking comprises ligating the unligated adapter to a blocking oligonucleotide via either a splint-dependent or a splint-independent ligation reaction to prevent circularization and concatamerization of the adapter. In some embodiments, the blocking oligonucleotide comprises at least one end blocking group to prevent its extension and ligation. In some embodiments, blocking of the unligated adapter comprises hybridizing circular or concatameric forms of the remaining unligated adapter with a blocking oligonucleotide partially complementary to the junction between the 5′ and 3′ ends of the adapter, wherein the blocking oligonucleotide prevents first primer extension and/or amplifying. In some embodiments, depleting comprises hybridizing circular or concatameric forms of the unligated adapter with a capture oligonucleotide partially complementary to the junction between the 5′ and 3′ ends of the adapter and capture of the resulting hybridized circular or concatameric forms of the adapter on a solid support. In some embodiments, degrading comprises degrading the unligated adapter before circularizing of the adapter-polynucleotide ligation product by an exonuclease. In each case, the degrading may be performed with an exonuclease are selected from: Exonuclease I, Exonuclease II associated with DNA polymerase I, Exonuclease III, Exonuclease VII, T5 Exonuclease, Terminator™ 5′-Phosphate-Dependent Exonuclease, Xm I exoribonuclease, Rec J exonuclease, RecJf, and RNAse R or combination thereof.

In some embodiments, the method further comprises purifying the adapter-polynucleotide ligation product before the circularizing. In some embodiments, the method comprises purifying an amplicon of the sequencing library.

In certain embodiments, the presence of small nucleic acids (e.g. RNAs) is detected and their amounts quantified by the miR-ID approach using the following steps: a) multiplex circularization of target nucleic acids using T4 RNA ligase 1 or CircLigase; b) multiplex synthesis of the corresponding multimers (MNA) by RT-RCA using a mixture of short (8-10 nt) target-specific RT primers and a reverse transcriptase; c) singleplex real-time qPCR using target-specific 5′-overlapping primers (which overlap for 13-19 nt at their 5′-ends and have 2-4 nt overhangs at their 3′ ends). An inexpensive, non-specific fluorescent dye such as SYBR Green or EVA Green can be used for signal detection. The qPCR step can be performed simultaneously for any number of different miRNAs under the same thermo-cycling conditions in the so-called “FOR array” or “virtual array” formats. These virtual multiplexing” techniques, which use physically separated FOR primers specific to different miRNAs, are easy to automate and can compete with true multiplex qPCR methods. Another variant of miR-ID, uses an isothermal technique instead of PCR for signal amplification (miRSA assay). This method shares the same circularization and RT-RCA steps with miR-ID, but differs in step c. In this last step, miRSA uses the isothermal, hyperbranched strand-displacement (HSDA) reaction rather than PCR, while both methods employ similar 5′-overlapping primer pairs.

In certain embodiments, described herein is a method of detecting a nucleic acid in a sample comprising circularizing the nucleic acid in the sample. The target nucleic acid may be circularized by ligating the 5′-end of the target nucleic acid to its 3′-end to produce a circularized target nucleic acid. In some embodiments, circularizing the target nucleic acid comprises use of a ligase selected from a T4 RNA ligase, a CircLigase, and a CircLigase II. The target nucleic acid may be any nucleic acid described herein. The target nucleic acid may be an RNA. The target nucleic acid may be derived from a dimorphic fungal organism. The target nucleic acid may be associated with a dimorphic fungal disorder.

In some embodiments, the methods comprise synthesizing a multimeric nucleic acid (MNA) comprising multiple repeats of sequences that are complementary to the target nucleic acid by rolling circle amplification. In some embodiments, synthesizing comprises hybridizing the circularized target nucleic acid with an oligonucleotide reverse transcriptase (RT) primer specific for the target nucleic acid. In some embodiments, synthesizing comprises enzymatically extending the oligonucleotide RT primer by a polymerase comprising reverse transcriptase activity to produce the MNA. In some embodiments, synthesizing comprises amplifying the MNA with a forward primer and a reverse primer to produce an amplicon. In some embodiments, the 3′-proximal region of the forward primer is complementary to a first portion of the multiple repeats within the MNA. In some embodiments, the 3′-proximal region of the reverse primer corresponds in sequence to a second portion of the multiple repeats within the MNA. In some embodiments, the forward primer and reverse primer can form a duplex with 3′ overhangs, wherein the duplex is shorter than the target sequence by at least 2 nucleotides. In some embodiments, the method comprises detecting the amplicon, thereby detecting the nucleic acid in the sample.

In some embodiments, the target nucleic acid comprises a 5′-phosphate (5′-p) or other 5′-end group that can be converted to 5′-p; a 3′-hydroxyl (3′-OH) or other 3′-end group that can be converted to 3′-OH; a 2′-OH or 2′-OMe at a 3′-terminal nucleotide; or a combination thereof. In some embodiments, the oligonucleotide RT primers has a sequence selected from a target-specific oligonucleotide sequence; and a fully or partially randomized oligonucleotide sequence of 6 nucleotides or more in length. In some embodiments, the oligonucleotide RT primer has a target-specific oligonucleotide sequence, wherein the target-specific oligonucleotide sequence is located on an array format or attached to target-designated beads. In some embodiments, the oligonucleotide RT primer is in solution, and the method further comprises, prior to detecting the MNA: non-covalently binding the MNA with a substantially complementary anchor oligonucleotide immobilized on a solid support to produce a captured MNA, and separating the captured MNA from irrelevant nucleic acid material.

In some embodiments, amplifying comprises isothermal strand displacement amplification. In some embodiments, amplifying comprises a thermocycling polymerase chain reaction. In some embodiments, a real-time qPCR signal is generated by a non-specific dye. In some embodiments, a real-time qPCR signal is generated upon extension of the forward PCR primer and reverse PCR primer, wherein the forward PCR primer comprises a fluorophore and the reverse PCR primer comprises a quencher, or the reverse PCR primer comprises a fluorophore and the forward PCR primer comprises a quencher. In some embodiments, the forward and reverse PCR primers comprise at each 3′ end a MNA-specific sequence and at least one additional sequence at each 5′ end selected from: a sequence which is neither substantially complementary to nor corresponding to any sequence present in the sample; a sequence encoding a Zip-code sequence; a sequence complementary to a universal signal-generating probe; and an additional adapter or linker sequence. In some embodiments, the method comprises PCR pre-amplification using the forward and reverse PCR primers, followed by real-time qPCR using an additional set of PCR primers. In some embodiments, the PCR primers are either both specific to the additional sequence; or comprise a first primer which is specific to the additional sequence and a second primer which is specific to the target sequence.

In some embodiments, the method comprises multiplex circularization, multiplex synthesis, multiplex amplification and/or multiplex detection of a plurality of target nucleic acids. The method may comprise detection of at least 5, 10, 15, 20, 25, 30, 40, 50, 60, 70, 80, 90, 100, 110, or 120 target nucleic acids described herein.

In some embodiments, a first portion and a second portion of each repeat of the multiple repeats at least partially overlaps. In some embodiments, the region of complementarity between the 3′-proximal region of the forward primer and the first portion of the multiple repeats within the MNA is between 16 and 21 nucleotides, and wherein the region of correspondence between the 3′-proximal region of the reverse primer and the second portion of the multiple repeats within the MNA is between 16 and 21 nucleotides.

### C. Methods of Treatment

In some embodiments, the methods described herein predict disease dissemination more accurately to advance earlier intervention and direct treatment. In some embodiments, the methods comprise identifying a patient at risk for a chronic dimorphic fungal disease. In some embodiments, the methods comprise identifying a subject afflicted with a chronic dimorphic fungal disease. In some embodiments, the methods comprise identifying a patient at risk for a disseminated dimorphic fungal disease. In some embodiments, the methods comprise identifying a subject afflicted with a disseminated dimorphic fungal disease. In some embodiments, the methods comprise identifying a subject afflicted with a disseminated dimorphic fungal disease and recommending treatment. In some embodiments, the methods comprise identifying a subject afflicted with a disseminated dimorphic fungal disease and treating the disseminated dimorphic fungal disease. In some embodiments, the methods comprise identifying a subject with residual dimorphic fungal disease. In some embodiments, the methods comprise treating a subject with a residual dimorphic fungal disease.

In some embodiments, the subject has or is suspected to have a dimorphic fungal disorder. In some embodiments, the subject has been diagnosed with a dimorphic fungal disorder. In some embodiments, the dimorphic fungal disorder is a chronic dimorphic fungal disorder. In some embodiments, the dimorphic fungal disorder is a disseminated dimorphic fungal disorder. In some embodiments, the dimorphic fungal disorder is a residual dimorphic fungal disorder. In some embodiments, the dimorphic fungal disease is selected from the list consisting of coccidioidomycosis (Valley Fever), histoplasmosis, blastomycosis, candidiasis, and combinations thereof. In some embodiments, the dimorphic fungal organism is selected from the list consisting of Coccidioides, Histoplasma capsulatum, Blastomyces, Candida, Lomentospora prolificans, and Scedosporum.

Coccidioidomycosis (Valley Fever) is a systemic infection caused by dimorphic fungi Coccidioides immitis and C. posadasii. Clinical manifestations range from mild flu-like disease to severe disseminated infection that can require life-long therapy. These soil-dwelling fungi are found in arid, desert-like conditions throughout the southwestern United States (primarily Arizona, California, Nevada, New Mexico, Texas and Utah), Mexico, Central and South America. In some embodiments, the dimorphic fungal disorder comprises Valley Fever. In some embodiments, the Valley Fever comprises chronic Valley Fever. In some embodiments, the Valley Fever comprises disseminated Valley Fever.

In some embodiments, the dimorphic fungal disorder results from an infection with a dimorphic fungal organism. In some embodiments, the dimorphic fungal organism is a Coccidiodes spp. In some embodiments, the Coccidiodes spp. is C. posadasii, C. immitis, or a combination thereof.

Histoplasmosis is a self-limiting infection with mild symptoms in a high percentage of immunocompetent patients. Unfortunately, for a significant number of immunocompromised patients, and for some immunocompetent patients there is an increased risk of developing systemic disease. The mortality rate in HIV infected persons with disseminated histoplasmosis disease is approximately 10%. The WHO estimates that disseminated histoplasmosis may be responsible for approximately 15% of AIDS-related deaths every year. Furthermore, histoplasmosis has the ability to remain in a quiescent stage for years and to reactivate when cell-mediated immunity is diminished or during treatment with immunosuppressive drugs. In some embodiments, the dimorphic fungal disorder comprises histoplasmosis. In some embodiments, the histoplasmosis comprises chronic histoplasmosis. In some embodiments, the histoplasmosis comprises disseminated histoplasmosis.

In some embodiments, the dimorphic fungal disorder results from an infection with a dimorphic fungal organism. In some embodiments, the dimorphic fungal organism comprises Histoplasma capsulatum.

Blastomycosis is a rare fungal infection usually acquired by breathing in the spores of the fungi Blastomyces dermatitidis or Blastomyces gilchristii. These fungi can be found in moist soils, particularly in wooded areas and along waterways. In some embodiments, the dimorphic fungal disorder comprises Blastomycosis. In some embodiments, the Blastomycosis comprises chronic Blastomycosis. In some embodiments, the Blastomycosis comprises disseminated Blastomycosis.

In some embodiments, the dimorphic fungal disorder results from an infection with a dimorphic fungal organism. In some embodiments, the dimorphic fungal organism is a Blastomyces spp. In some embodiments, the Blastomyces spp. is Blastomyces dermatitidis, B. gilchristii, or a combination thereof.

Candidiasis usually affects the skin and/or the mucous membranes of the mouth, intestines, or the vagina. Candida infections are rarely serious in otherwise healthy people. In rare cases, it may spread through other parts of the body, such as when a patient is receiving chemotherapy or broad spectrum antibiotics. In the most severe cases it can affect the blood, the membrane lining the heart muscle (endocardium), or membranes around the brain (meninges). In some embodiments, the dimorphic fungal disorder comprises candidiasis. In some embodiments, the Candidiasis comprises chronic candidiasis. In some embodiments, the Blastomycosis comprises disseminated candidiasis.

In some embodiments, the dimorphic fungal disorder results from an infection with a dimorphic fungal organism. In some embodiments, the dimorphic fungal organism is a Candida spp. In some embodiments, the Candida spp. is C. albicans, C. auris or a combination thereof.

### Therapeutics

In some embodiments, the subject is treated with an anti-fungal treatment. In some embodiments, the subject is treated based on a presence, absence, or quantity of at least one of any one of SEQ ID NO: 1-120 detected in a sample from the subject. In some embodiments, the subject is treated based on the results of an assay described herein. In some embodiments, a treatment is selected for the subject based on a presence, absence, or quantity of at least one of any one of SEQ ID NO: 1-120. In some embodiments, the treatment is selected for the subject based on the results of an assay described herein.

In some embodiments, the anti-fungal treatment comprises an amphotericin B compound (AmB). In some embodiments the AmB comprises intrathecal AmBd or L-AmB. In some embodiments, the anti-fungal treatment comprises an azole. In some embodiments, the azole comprises Fluconazole, Itraconazole, SUBA-itraconazole, Voriconazole, Posaconazole, Isavuconazonium, or a combination thereof. In some embodiments, the anti-fungal treatment is Olorofim.

### II. Kits and Compositions

### Compositions

Disclosed herein are compositions useful for the detection of a sequence in a sample obtained from a subject according to the methods described herein. Aspects disclosed herein provide compositions comprises a polynucleotide sequence comprising at least 10 but less than 50 contiguous nucleotides of any one of SEQ ID NOS: 1-120 or reverse complements thereof, wherein the contiguous polynucleotide sequence comprises a detectable molecule. In various embodiments, the detectable molecule comprises a fluorophore. In other embodiments, the polynucleotide sequences further comprise a quencher.

In some embodiments, the compositions comprises an adapter. In some embodiments, the adapter is in the form of a single-stranded nucleic acid comprising: a) a single-stranded 5′-proximal segment and a single-stranded 3′-proximal segment, wherein the single-stranded 5′-proximal segment and the single-stranded 3′ proximal segment comprise a sequencing adapter, a detection sequence, or a combination thereof; b) 5′-end and 3′-end groups that allow first and second consecutive ligation reactions either directly or after conversion of one or both 5′-end and 3′-end groups to ligatable 5′-end and ligatable 3′-end group(s); and c) a template-deficient segment linking the single-stranded 5′ proximal segment and the single-stranded 3′ proximal segment that restricts primer extension by a polymerase over the template-deficient segment. In some embodiments, the adapter comprises at least one DNA residue and at least one DNA residue. In some embodiments, the adapter comprises at least one RNA residue, one DNA residue, modified nucleic acid residue, or non-nucleotide residue, or a combination thereof. In some embodiments, the adapter comprises at least one sequence selected from: a sequencing adapter, a primer binding site, a detection sequence, a probe hybridization sequence, a capture oligonucleotide binding site, a polymerase binding site, an endonuclease restriction site, a sequencing bar-code, an indexing sequence, a Zip-code, one or more random nucleotides, a unique molecular identifier (UMI), and a sequencing flow-cell binding site, and combinations thereof. In some embodiments, the 5′-end and/or 3′-end of the adapter contains a reversible blocking group preventing circularization or concatamerization of the adapter while allowing its ligation with a sample polynucleotide, wherein the reversible blocking group requires chemical, photochemical or enzymatic conversion to an active end group prior to the circularization of the adapter-polynucleotide ligation product. In some embodiments, the reversible blocking group is a 3′-end-blocking group selected from: 3′-p, 2′,3′-cyclic phosphate (2′,3′>p), 3′-O-(α-methoxyethyl) ether, and 3′-O-isovaleryl ester.

### Kits

Disclosed herein, are kits useful for to detect the genotypes and/or biomarkers disclosed herein. In some embodiments, the kits disclosed herein may be used to diagnose and/or treat a disease or condition in a subject; or select a patient for treatment and/or monitor a treatment disclosed herein. In some embodiments, the kit comprises the compositions described herein, which can be used to perform the methods described herein. Kits comprise an assemblage of materials or components, including at least one of the compositions. Thus, in some embodiments the kit contains a composition including of the pharmaceutical composition, for the treatment of the dimorphic fungal disorder. In other embodiments, the kits contains all of the components necessary and/or sufficient to perform an assay for detecting and measuring dimorphic fungal disorder markers, including all controls, directions for performing assays, and any necessary software for analysis and presentation of results. In some embodiments, the kit comprises an RNA buffer, an adapter, an RNAse Inhibitor, a ligation buffer, a ligase, a blocking agent, a blocking enzyme, a dimer removal agent, a reverse transcriptase primer, a dNTP, a reverse transcriptase, a PCR polymerase, a miRNA control, or a combination thereof.

In some instances, the kits described herein comprise components for detecting the presence, absence, and/or quantity of a target nucleic acid described herein. In some embodiments, the kit comprises the compositions (e.g., primers, probes) described herein. The disclosure provides kits suitable for assays such as NGS, PCR, and qPCR. In some embodiments, the kit comprises an adapter as described herein. In some embodiments, the kit comprises the exact nature of the components configured in the kit depends on its intended purpose.

In some embodiments, the kits described herein are configured for the purpose of treating and/or characterizing a disease or condition (e.g., dimorphic fungal disease), or subclinical phenotype thereof (e.g. acute, chronic or disseminating disease phenotypes) in a subject. In some embodiments, the kits described herein are configured for the purpose of identifying a subject suitable for treatment with an antifungal (e.g. an azole). In some embodiments, the kit is configured particularly for the purpose of treating mammalian subjects. In some embodiments, the kit is configured particularly for the purpose of treating human subjects. In further embodiments, the kit is configured for veterinary applications, treating subjects such as, but not limited to, farm animals, domestic animals, and laboratory animals. In some embodiments, the kit is configured to select a subject for a therapeutic agent, such as those disclosed herein. In some embodiments, the kit is configured to select a subject for treatment with a therapeutic agent disclosed herein. An exemplary therapeutic agent is an azole.

Instructions for use may be included in the kit. Optionally, the kit also contains other useful components, such as, diluents, buffers, pharmaceutically acceptable carriers, syringes, catheters, applicators, pipetting or measuring tools, bandaging materials or other useful paraphernalia. The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as compositions and the like. The packaging material is constructed by well-known methods, preferably to provide a sterile, contaminant-free environment. The packaging materials employed in the kit are those customarily utilized in gene expression assays and in the administration of treatments. As used herein, the term “package” refers to a suitable solid matrix or material such as glass, plastic, paper, foil, and the like, capable of holding the individual kit components. Thus, for example, a package can be a glass vial or prefilled syringes used to contain suitable quantities of the pharmaceutical composition. The packaging material has an external label which indicates the contents and/or purpose of the kit and its components.

### III. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

Non-limiting examples of a “biological sample” include any animal cells, tissues, or fluids from which nucleic acids and/or proteins can be obtained. As non-limiting examples, this includes whole blood, peripheral blood, plasma, serum, saliva, mucus, urine, semen, lymph, fecal extract, cheek swab, cells or other bodily fluid or tissue, including but not limited to tissue obtained through surgical biopsy or surgical resection. Alternatively, a sample can be obtained through primary patient derived cell lines, or archived patient samples in the form of preserved samples, or fresh frozen samples.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

As used herein, the phrase “substantially identical,” or “substantial identity” in the context of two nucleic acid molecules, nucleotide sequences or protein sequences, refers to two or more sequences or subsequences that have at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and/or 100% nucleotide or amino acid residue identity, when compared and aligned for maximum correspondence, as measured using one of the following sequence comparison algorithms or by visual inspection. In some embodiments, substantial identity refer to two or more sequences or subsequences that have at least about 70%, at least about 75%, at least about 80%, at least about 85%, at least about 90%, at least about 95, 96, 97, 98, or 99% identity. For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, CA). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences is to a full-length polynucleotide sequence or to a portion thereof, or to a longer polynucleotide sequence. In some instances, “Percent identity” or “sequence identity” is determined using BLASTX version 2.0 for translated nucleotide sequences and BLASTN version 2.0 for polynucleotide sequences. Percent identity may refer to either a given sequence or the reverse complement thereof.

As described herein, the term “dimorphic fungal disorders”, also referred interchangeably as a “dimorphic fungal disease” or “dimorphic fungal infection” may refer to a disorder caused by a dimorphic fungal pathogen. A dimorphic fungal pathogen is a fungal pathogen that is capable of changing its morphological form from mold to yeast. In some circumstances, the fungal pathogen may grow in a first morphological form at first (e.g. warmer) temperatures and a second morphological form at a second (e.g. colder) temperature.

As described herein, a “disseminated dimorphic fungal disease” may refer to a disseminated dimorphic fungal disorder with symptoms in at least one tissue outside of the lungs. The tissue may be the skin, brain, heart, bones, liver, or meninges. The terms “disseminated dimorphic fungal disease” and “disseminated dimorphic fungal disorder” may be used interchangeably. As defined herein, a “residual dimorphic fungal disease” may comprise a dimorphic fungal disease or disorder that persists after treatment has been provided to the subject. The terms “residual dimorphic fungal disease” and “residual dimorphic fungal disorder” may be used interchangeably.

The term “small RNA,” as used herein, may refer to a RNA molecule or RNA fragment that is at least about 10 nucleotides to at least about 200 nucleotides. A small RNA may be a fragment of a longer RNA. A small RNA may lack at least one feature of a mature RNA such as a polyadenosine tail or a 5′ cap, or a combination thereof. A small RNA may be a noncoding RNA. Nonlimiting examples of small RNA include microRNA, piwi-interacting RNA, small interfering RNA, small nuclear RNA, small nucleolar RNA, or combinations thereof.

The section headings used herein are for organizational purposes only and are not to be construed as limiting the subject matter described.

### IV. Numbered Embodiments

Also disclosed herein are the following embodiments:

Embodiment 1 comprises a method of treating a dimorphic fungal disorder in a subject in need thereof, the method comprising: obtaining a biological sample from the subject; detecting a nucleic acid; and determining that the subject is afflicted with a chronic disease associated with a dimorphic fungal organism. Embodiment 2 comprises the method of embodiment 1, further comprising determining that the subject is afflicted with a disseminating disease. Embodiment 3 comprises the method of embodiment 1 or 2, wherein the dimorphic fungal disorder is selected from the list consisting of coccidioidomycosis, histoplasmosis, blastomycosis, candidiasis or a combination thereof. Embodiment 4 comprises the method of any one of embodiments 1-3, wherein the dimorphic fungal disorder comprises coccidioidomycosis. Embodiment 5 comprises the method of any one of embodiments 1-3, wherein the dimorphic fungal disorder comprises histoplasmosis. Embodiment 6 comprises the method of any one of embodiments 1-3, wherein the dimorphic fungal disorder comprises blastomycosis. Embodiment 7 comprises the method of any one of embodiments 1-3, wherein the dimorphic fungal disorder comprises candidiasis. Embodiment 8 comprises the method of any one of embodiments 1-7, wherein the dimorphic fungal disorder results from an infection with a dimorphic fungal organism. Embodiment 9 comprises the method of any one of embodiments 1-8, wherein the dimorphic fungal organism is selected from the list consisting of coccidioides, Histoplasma capsulatum, blastomyces, or candida. Embodiment 10 comprises the method of embodiment 8 or 9, wherein the dimorphic fungal organism comprises a coccidioides spp. Embodiment 11 comprises the method of embodiment 8 or 9, wherein the dimorphic fungal organism comprises Histoplasma capsulatum. Embodiment 12 comprises the method of embodiment 8 or 9, wherein the dimorphic fungal organism comprises a blastomyces spp. Embodiment 13 comprises the method of embodiment 8 or 9, wherein the dimorphic fungal organism comprises a candida spp. Embodiment 14 comprises the method of any one of embodiments 1-12, wherein the nucleic acid is derived from the dimorphic fungal organism. Embodiment 15 comprises the method of any one of embodiments 1-14, wherein the nucleic acid comprises an RNA. Embodiment 16 comprises the method of embodiment 15, wherein the RNA is no more than 100, 90, 80, 70, 60, 50, 40, 30, 20, or 10 nucleotides in size. Embodiment 17 comprises the method of any one of embodiments 1-16, wherein the biological sample is selected from the list consisting of plasma, saliva, serum, and urine. Embodiment 18 comprises the method of any one of embodiments 1-17, wherein the biological sample comprises plasma. Embodiment 19 comprises the method of any one of embodiments 1-17, wherein the biological sample comprises saliva. Embodiment 20 comprises the method of any one of embodiments 1-17, wherein the biological sample comprises serum. Embodiment 21 comprises the method of any one of embodiments 1-17, wherein the biological sample comprises urine. Embodiment 22 comprises the method of any one of embodiments 1-22, wherein the nucleic acid is detected by a nucleic acid amplification reaction. Embodiment 23 comprises the method of any one of embodiments 1-22, wherein the nucleic acid is detected by qPCR. Embodiment 24 comprises the method of any one of embodiments 1-23, further comprising treating with an antifungal. Embodiment 25 comprises the method of embodiment 24, wherein the antifungal is selected from the list comprising a amphotericin B compound and an azole. Embodiment 26 comprises the method of embodiment 25, wherein the azole comprises Fluconazole.

Embodiment 27 comprises a method of diagnosing a chronic disease in a subject with a dimorphic fungal infection, the method comprising: obtaining a biological sample from the subject; detecting a nucleic acid; determining that the subject is afflicted with a chronic disease associated with a dimorphic fungal organism; and treating the subject an antifungal effective to treat the chronic disease associated with the dimorphic fungal organism. Embodiment 28 comprises the method of embodiment 27, further comprising determining that the subject is afflicted with a disseminating disease. Embodiment 29 comprises the method of embodiment 27 or 28, wherein the dimorphic fungal disorder is selected from the list consisting of coccidioidomycosis, histoplasmosis, blastomycosis, candidiasis, and combinations thereof. Embodiment 30 comprises the method of any one of embodiments 27-29, wherein the dimorphic fungal disorder comprises coccidioidomycosis. Embodiment 31 comprises the method of any one of embodiments 27-30, wherein the dimorphic fungal disorder comprises histoplasmosis. Embodiment 32 comprises the method of any one of embodiments 27-30, wherein the dimorphic fungal disorder comprises blastomycosis. Embodiment 33 comprises the method of any one of embodiments 27-30, wherein the dimorphic fungal disorder comprises candidiasis. Embodiment 34 comprises the method of any one of embodiments 27-32, wherein the dimorphic fungal disorder results from an infection with a dimorphic fungal organism. Embodiment 35 comprises the method of any one of embodiments 24-34, wherein the dimorphic fungal organism is selected from the list consisting of coccidioides, Histoplasma capsulatum, blastomyces, or candida. Embodiment 36 comprises the method of embodiment 34 or 35, wherein the dimorphic fungal organism comprises a coccidioides spp. Embodiment 37 comprises the method of embodiment 34 or 35, wherein the dimorphic fungal organism comprises Histoplasma capsulatum. Embodiment 38 comprises the method of embodiment 34 or 35, wherein the dimorphic fungal organism comprises a blastomyces spp. Embodiment 39 comprises the method of embodiment 34 or 35, wherein the dimorphic fungal organism comprises a candida spp. Embodiment 40 comprises the method of any one of embodiments 24-39, wherein the nucleic acid is derived from the dimorphic fungal organism. Embodiment 41 comprises the method of any one of embodiments 27-40, wherein the nucleic acid comprises an RNA. Embodiment 42 comprises the method of embodiment 41, wherein the RNA is no more than 100, 90, 80, 70, 60, 50, 40, 30, 20, or 10 nucleotides in size. Embodiment 43 comprises the method of any one of embodiments 27-42, wherein the biological sample is selected from the list consisting of plasma, saliva, serum, and urine. Embodiment 44 comprises the method of any one of embodiments 27-42, wherein the biological sample comprises plasma. Embodiment 45 comprises the method of any one of embodiments 27-42, wherein the biological sample comprises saliva. Embodiment 46 comprises the method of any one of embodiments 27-42, wherein the biological sample comprises serum. Embodiment 47 comprises the method of any one of embodiments 27-42, wherein the biological sample comprises urine. Embodiment 48 comprises the method of any one of embodiments 27-47, wherein the nucleic acid is detected by amplification. Embodiment 49 comprises the method of any one of embodiments 27-48, wherein the nucleic acid is detected by qPCR. Embodiment 50 comprises the method of any one of embodiments 27-49, further comprising treating with an antifungal. Embodiment 51 comprises the method of embodiment 50, wherein the antifungal is selected from the list comprising an amphotericin B compound and an azole. Embodiment 52 comprises the method of embodiment 51, wherein the azole comprises Fluconazole.

### V. Examples

The following examples are included for illustrative purposes only and are not intended to limit the scope of the invention.

### Example 1: A Test to Detect Disseminated Fungal Infections

A biological sample (e.g. saliva, urine, plasma or serum) is isolated from a subject with a fungal infection to determine if the subject is at risk of developing a disseminated fungal infection. Nucleic acids are isolated from the biological sample and amplified (e.g. through qPCR). Target nucleic acids derived from fungal pathogens are detected in the sample. The subject is identified as being at risk of developing disseminated fungal infection. The subject is administered antifungal drugs.

### Example 2: Valley Fever Patient Samples

Samples from 56 Valley Fever positive patients and 10 negative donors were collected. Serum samples were preserved with RNA/DNA Shield (Zymo Research) with a 1:1 ratio ensuring samples were biosafe and stabilizing both RNA and DNA molecules. Samples were used to extract circulating RNA using Zymo Quick-cfRNA kits as recommended by the manufacturer with an input serum of 200 μL per extraction. For this preliminary experiment, extracted RNA was used to prepare sequencing libraries for canonical small RNAs (with 5′P and 3′OH) with RealSeq-Biofluids. Sequencing libraries for each sample were individually barcoded and sequenced on an Illumina NextSeq 550, to a coverage of >10 million reads (75 nt lengths). Sequencing reads for all samples were trimmed of adapters. After preliminary analysis of the sequencing data from patients with disseminated infection, 22 highly expressed ncRNAs were identified from the Coccidioides immitis genome (Valley Fever), this set of ncRNAs was used as reference for further analysis. Sequencing data from all samples, including negative controls and patients both with localized and disseminated infection, were aligned to the 22 highly expressed ncRNAs.

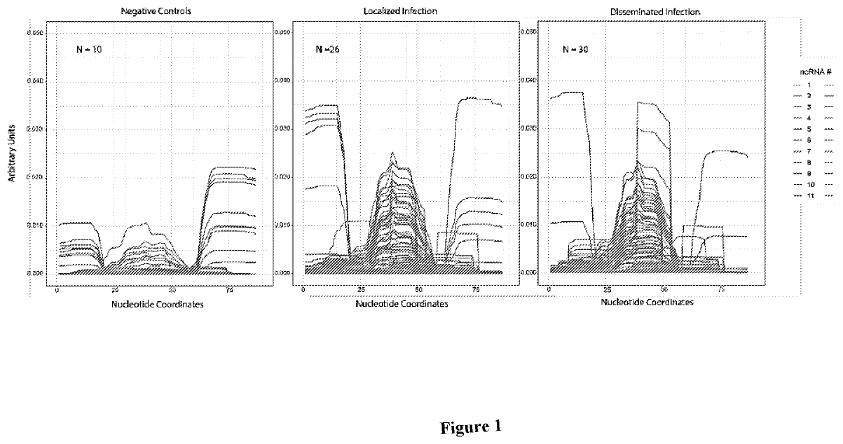

Results from those alignments are shown on FIG. 1. While the average length for the ncRNAs included in the database is 90nt, most of the reads identified are only around 20-25nt (FIG. 1), showing high fragmentation of the mature ncRNAs. Interestingly, many of the 22 ncRNAs used for the analysis are known to have RNA modifications, with the regions including the modifications having significantly lower numbers of sequencing reads (data not shown). It has been widely reported that RNA modifications can interfere with the reverse transcription reaction and inhibit the detection of modified RNAs with standard techniques (19). Despite the short length of the reads mapping to the VF-specific ncRNAs there is a clear enrichment for regions of specific and distinct ncRNAs for patients with positive infection.

### Example 3: Identifying Biomarkers of Infection

An index of Coccidioides genomic regions that are present as RNA fragments was created. A ‘Cocci Index’ was generated by aligning small RNA sequencing publicly available data (PMID 35076277) to the Coccidioides genome. Genomic regions with a low rate of alignment were removed to obtain a high confidence ‘Cocci Index’.

RNA from serum samples from Valley Fever patients and negative donors was used to prepare sequencing libraries using RealSeq®-Biofluids. Libraries were sequenced with an Illumina NextSeq 550. Raw reads from both patients and controls were aligned to the ‘Cocci Index’ and regions with differential expression between both groups were identified as markers of infection (120 genomic regions were identified, Table 2)

TABLE 2
Differentially expressed genes

| SEQ ID | |
|---|---|
| NO | Sequence |
| 1 | TGCGGAAGGATCATTA |
| 2 | CTGCGGAAGGATCATTA |
| 3 | AATAAGGATTGACAGATTGAGAGCTCTTT |
| 4 | GTCGAGTTGTTTGGGAATGCAGCT |
| 5 | ATAAGGATTGACAGATTGAGAGCTCTTT |
| 6 | CGATGGAAGTTTGAGGC |
| 7 | GTTCTTAGTTGGTGGAGT |
| 8 | AAGGATTGACAGATTGAGAGCTCTTT |
| 9 | AATAAGGATTGACAGATTGAGAGCTCTTTCTT |
| 10 | GCGGAAGGATCATTA |
| 11 | ATAAGGATTGACAGATTGAGAGCTCTTTC |
| 12 | ATTAGTGACGCGCATGAATG |
| 13 | AATAAGGATTGACAGATTGAGAGCTCTTTC |
| 14 | TTAGTGACGCGCATGAATG |
| 15 | TAGTTTGACATTGTGAAAAGAC |
| 16 | AATTAGTGACGCGCATGAATG |
| 17 | CGTTCTTAGTTGGTGGAGT |
| 18 | TTCGGGTCTTGTAATTGGAATGAGTACA |
| 19 | AAATAAGGATTGACAGATTGAGAGCTCTTT |
| 20 | AGGGGTGTATTTATTAGATA |
| 21 | CTCTAGTGCAGATCTTGGTGGTAGTAGC |
| 22 | GCCGGAAAGTTGGTCAAACTTGGTCATT |
| 23 | TAAGGATTGACAGATTGAGAGCTCTTT |
| 24 | TCGACGAGTCGAGTTGTTTGGGAATGCAGC |
| 25 | TCGACGAGTCGAGTTGTTTGGGA |
| 26 | TAAGGGAAGTCGGCAAAATAGATCC |
| 27 | AAGGATTGACAGATTGAGAGCTCT |
| 28 | CTCTAGTTTGACATTGTGAAAAGAC |
| 29 | AATAAGGATTGACAGATTGAGAGCTCT |
| 30 | ATAAGGATTGACAGATTGAGAGCTCT |
| 31 | AGCAGAATTCGGTAAGC |
| 32 | AAATAAGGATTGACAGATTGAGAGCTCTTTC |
| 33 | AGGACGTCATAGAGGGTGAGAATCCC |
| 34 | AGGTGAAATTCTTGGATTTGCTGAAGACT |
| 35 | AAGGATTGACAGATTGAGAGC |
| 36 | CGAGTCGAGTTGTTTGGGAATGCAGCT |
| 37 | ACAGGACGTCATAGAGGGTGAGAATCCCGTCT |
| 38 | TTAGTGACGCGCATGAATGGATT |
| 39 | ATCAACTTTCGATGGTAGGA |
| 40 | TTAACGAGGAACAATTGGAGGGC |
| 41 | TCGACGAGTCGAGTTGTTTGGGAATGCAGCTCT |
| 42 | TATCAACTTTCGATGGTAGGA |
| 43 | AAAATAAGGATTGACAGATTGAGAGCTCTTT |
| 44 | TTGTCGAATCGCATGGCCTTGCGCTGGC |
| 45 | AGGACGTCATAGAGGGTGAGAATCCCG |
| 46 | CTCTAGTTTGACATTGTGAAAAG |
| 47 | TGAGGTAAAGCGAATGATT |
| 48 | TCCTTGGAACAGGACGTCATAGA |
| 49 | CAGGACGTCATAGAGGGTGAGAATCCCGTCT |
| 50 | ATAAGGATTGACAGATTGAGAGC |
| 51 | TAAGGATTGACAGATTGAGAGC |
| 52 | AGAATTCGGTAAGCGTTGGATTGTT |
| 53 | TCTAGTTTGACATTGTGAAAAGAC |
| 54 | TTCGACGAGTCGAGTTGTTTGGGAATGCAGC |
| 55 | TAATTAGTGACGCGCATGAATG |
| 56 | TCTTCGGATGGATTTGAGTAAGA |
| 57 | AATAAGGATTGACAGATTGAGAGC |
| 58 | AAGCAGAATTCGGTAAGC |
| 59 | TATGAGGTAAAGCGAATGATT |
| 60 | AGTGAACGAAAGTTAGGGGATC |
| 61 | TCGACGAGTCGAGTTGTTTGGGAATGCAGCTCTAAGTGGGT |
| 62 | AAAATAAGGATTGACAGATTGAGAGCTCTTTC |
| 63 | TCGACGAGTCGAGTTGTTTGGGAATGCAGCTC |
| | TAAGTGGGTGGT |
| 64 | AGGTGAAATTCTTGGATTTGCTGAAGACTAAC |
| 65 | ATCGAAGACGATCAGATAC |
| 66 | ACAGGACGTCATAGAGGGTGAGAATCCC |
| 67 | AAGGGGTGTATTTATTAGAT |
| 68 | AGGGGTGTATTTATTAGAT |
| 69 | TAAGGATTGACAGATTGAGAGCTCT |
| 70 | AATGATTAATAGGGATAGTCGGG |
| 71 | AAGGATTGACAGATTGAGAGCTC |
| 72 | TTCGACGAGTCGAGTTGTTTGGGA |
| 73 | AAGGATTGACAGATTGAGAGCT |
| 74 | GATGGTAGGATAGTGGCCT |
| 75 | TCGACGAGTCGAGTTGTTTGGGAATGCAGCT |
| 76 | TAAGTTCCTTGGAACAGGACGTCATAGAG |
| 77 | CGGGAAAAGGATTGGCTC |
| 78 | CGAGTAGGCAGGCGTGGAGGTT |
| 79 | TTGTTTGGGAATGCAGCTCTA |
| 80 | AATAAGGATTGACAGATTGAGA |
| 81 | ATGAGGTAAAGCGAATGATT |
| 82 | TCGATGGTAGGATAGTGGCC |
| 83 | AGCGAATGATTAGAGGC |
| 84 | AAATAAGGATTGACAGATTGAGAGCTCT |
| 85 | ATAAGGATTGACAGATTGAGAGCT |
| 86 | AACTTTCGATGGTAGGATA |
| 87 | ATAAGGATTGACAGATTGAGAGCTC |
| 88 | GCGAATGATTAGAGGCCTTGGGGTT |
| 89 | GACGAGTAGGCAGGCGTGGAGGTT |
| 90 | TCGACGAGTCGAGTTGTTTGGGAATG |
| 91 | TTTCGATGGTAGGATAGTGGCCT |
| 92 | GAAAGTTAGGGGATCGAAGACGATC |
| 93 | TTCGATGGTAGGATAGTGGCC |
| 94 | CGTGCAAATCGATCGTCAAA |
| 95 | GTCGAGTTGTTTGGGAATGCAGCTCTAAGTGGGT |
| 96 | AGGGAAGTCGGCAAAATAGATC |
| 97 | AGGTGAAATTCTTGGATT |
| 98 | TTCGATGGTAGGATAGTGGCCT |
| 99 | AATAAGGATTGACAGATTGAGAGCTC |
| 100 | TGAAAATGGATGGCGCTCAAGC |
| 101 | AATAAGGATTGACAGATTGAGAGCT |
| 102 | TCGATGGTAGGATAGTGGCCT |
| 103 | TTTCGATGGTAGGATAGTGGCC |
| 104 | ACTTTCGATGGTAGGATA |
| 105 | TCGACGAGTCGAGTTGTTTGGGAAT |
| 106 | GTTCTTAGTTGGTGGAGTG |
| 107 | AGGTAAAGCGAATGATT |
| 108 | CGACGAGTAGGCAGGCGTGGA |
| 109 | CGACGAGTAGGCAGGCGTGGAGGTT |
| 110 | CTTTCGATGGTAGGATAGTG |
| 111 | ACAGCCTCTAGTGCAGATCTTGGTGGTAGTAGC |
| 112 | GTTGTTTGGGAATGCAGCTCTA |
| 113 | AGGGATAACTGGCTTGT |
| 114 | AGGACGTCATAGAGGGTGAGAATCCCGTCTTTG |
| 115 | GTTAGGGGATCGAAGACGATC |
| 116 | AAATAAGGATTGACAGATTGAGAGCTCTTTCTT |
| 117 | CAGGACGTCATAGAGGGTGAGAATCCC |
| 118 | AGGGAAGCGCTTGCAACCAGACTCGGTCGTGGG |
| 119 | ACTTTCGATGGTAGGATAGTG |
| 120 | TAATAGGGATAGTCGGGGGC |

Coccidioides genomic regions that are present as small RNAs but that do not signal infection were also identified. These regions are non-specific markers, that map to the Coccidioides genome, can be detected in human samples, but that are not indicative of infection (158 genomic regions were identified (Table 3))

TABLE 3
Non-specific markers

| SEQ | |
|---|---|
| ID | |
| NO | Sequence |
| 121 | AAAGCTCGTAGTTGAACCTTGGGTCTGGCTGGCC |
| 122 | AACTTAGAACTGGTACGGACAAGGGGA |
| 123 | TTTGACCTCGGATCAGGTAGGAGTACCCGCTGAACTTAA |
| 124 | ATTCGGTAAGCGTTGGATTGTT |
| 125 | TTTGACCTCGGATCAGGTAGGAGTACCCGCTGAAC |
| 126 | AACTTAGAACTGGTACGGACAAGGGGAATCTGACTGTCT |
| 127 | AATTCGGTAAGCGTTGGATTGTTCACC |
| 128 | TTTGACCTCGGATCAGGTAGGAGTACCCGCTGAA |
| 129 | TGATAGAACAATGTAGATAAGGGAAGTCGGC |
| 130 | TGGCTTCTTAGAGGGACTATCGGCTCAAGCCGAT |
| 131 | GAACTTTGAGGACTGAAGTGGGGAAAGGTTCCATGT |
| 132 | TTCCGGGGGGCATGCCTGTTCGAGCGTC |
| 133 | TTTGACCTCGGATCAGGTAGGAGTACCC |
| 134 | GTACGGTGAAACTGCGAATGGCTCATTAAATC |
| 135 | CCACTAATAGGGAACGTGAGCT |
| 136 | GAACTGGTACGGACAAGGGGAATCTGACTGTCT |
| 137 | TAGAACTGGTACGGACAAGGGGAATCTGACTGTCT |
| 138 | AACAATGTAGATAAGGGAAGTC |
| 139 | TTTGACCTCGGATCAGGTAGGAGTACCCGCTG |
| 140 | CGATAGCGCACAAGTAGAGTGATC |
| 141 | GGTTCTATTTTGTTGGTTTCTAGGAC |
| 142 | TTAGAACTGGTACGGACAAGGGGAATCTGACTGTC |
| 143 | ACTTAGAACTGGTACGGACAAGGGGAATCTGACTGTC |
| 144 | CAATAAATACTGATACAGGGC |
| 145 | TACCTGGTTGATTCTGCCAGTAGTCATATGCTTGTC |
| 146 | TTTGACCTCGGATCAGGTAGGAGTACCCGCTG |
| | AACTTAAGCATATCAATAAGC |
| 147 | TTCGGTAAGCGTTGGATTGTTCACCCACT |
| 148 | AGAACTGGTACGGACAAGGGGAATCTGACTGTCT |
| 149 | CCAAGGATGTTTTCATTAATCAGTGAAC |
| 150 | TTTGACCTCGGATCAGGTAGGAGTACCCGCTGAAC |
| | TTAAGCATATCA |
| 151 | GAATTCGGTAAGCGTTGGATTGTTCACCC |
| 152 | GAATAATAGAATAGGACGTGT |
| 153 | GTTTGACCTCGGATCAGGTAGGAGTACCCGCT |
| 154 | GACAATAAATACTGATACAGGGCTC |
| 155 | ATGGAATAATAGAATAGGACGTGT |
| 156 | AATCGATCGTCAAATTTGGGTATAGGGGC |
| 157 | CACCCACTAATAGGGAACGTGAGCT |
| 158 | GTGCAGATCTTGGTGGTAGTAGCA |
| 159 | TTTGACCTCGGATCAGGTAGGAGTACCCGC |
| 160 | AGTTGATAGAACAATGTAGATAAGGGAAGTCGGC |
| 161 | GAGAACTTTGAGGACTGAAGTGGGGAAAGGTTCCATG |
| 162 | GATAGAACAATGTAGATAAGGGAAGTCGGC |
| 163 | TTTGACCTCGGATCAGGTAGGAGTA |
| 164 | TCGCCAAGGATGTTTTCATTAATCAGTGAAC |
| 165 | ACTTAGAACTGGTACGGACAAGGGGA |
| 166 | ACTTAGAACTGGTACGGACAAGGGGAATCTGACTGTCT |
| 167 | CGGGGGGCATGCCTGTTCGAGCGTC |
| 168 | TACGGTGAAACTGCGAATGGCTCATTAAATC |
| 169 | AAGTCGGAATCCGCTAAGGAGTGTGTA |
| 170 | TTTGACCTCGGATCAGGTAGGAGTACCCGCTGAACTTAAGCATA |
| 171 | TGAAACTTAAAGAAATTGACGG |
| 172 | AGGATTGGCTCTAAGGGTCGGGC |
| 173 | CTGTTCGAGCGTCATT |
| 174 | GTGACAATAAATACTGATACAGGGCTC |
| 175 | ATAGCGACGTTGCTTTTTGAT |
| 176 | TAAGCGTTGGATTGTTCACCCACT |
| 177 | TTTTTGATCCTTCGATGTCGGCTCTTC |
| 178 | GACCAACTTAGAACTGGTACGGACA |
| 179 | TGGGACCCGAAAGATGGTGAACTATGCC |
| 180 | TAGTTGATAGAACAATGTAGATAAGGGAAGTCGGC |
| 181 | AACTTAGAACTGGTACGGACAAGGGGAAT |
| 182 | AAGCGGAGGAAAAGAAACCAAC |
| 183 | AAGGATGTTTTCATTAATCAGTGAAC |
| 184 | GGGACCCGAAAGATGGTGAACTATGCC |
| 185 | ATGGATCGTTACTAGTGGGCCATTTTTG |
| 186 | ACCCACTAATAGGGAACGTGAGCT |
| 187 | AGAACTTTGAGGACTGAAGTGGGGAAAGGTTCCATGT |
| 188 | ATAAGCGGAGGAAAAGAAACCAAC |
| 189 | TGGTGAACTATGCCTGAATAGGGT |
| 190 | ATCGATCGTCAAATTTGGGTATAGGGGCG |
| 191 | CAGGTCTGTGATGCCCTTAGATG |
| 192 | TACCTGGTTGATTCTGCCAGTAGTCATATGCTTGT |
| 193 | GTTGGGACCCGAAAGATGGTGAAC |
| 194 | GAAAAGCCTCTAGTTGATAGAACAATGTAG |
| 195 | CGTGGAGCCTGCGGCTTA |
| 196 | ACTTAGAACTGGTACGGACAAGGGGAATCTGACTGTCTA |
| 197 | TAGTACGAGAGGAACCGTTGATTCAGATA |
| 198 | GGATTCTCCACGGCAACGTAACTGAACGCGGA |
| 199 | GAAAAGCCTCTAGTTGATAGAACAATG |
| 200 | GAAAAGGATTGGCTCTAAGGGT |
| 201 | ATTCCGGGGGGCATGCCTGTTCGAGCGTC |
| 202 | CGCCAAGGATGTTTTCATTAATCAGTGAAC |
| 203 | TTTGACCTCGGATCAGGTAGGAGTACCCGCTGA |
| 204 | ATAGCGACGTTGCTTTTTGATC |
| 205 | CAAATGAGAACTTTGAGGACTGAAGTG |
| 206 | CAACTTAGAACTGGTACGGACAAGGGG |
| 207 | GAAAGAAGACCCTGTTGAGCTTGACTC |
| 208 | AAGCGTTGGATTGTTCACCCACTAATA |
| 209 | TAGAACTGGTACGGACAAGGGGA |
| 210 | GGTTTGACCTCGGATCAGGTAGGAGTACCCGCT |
| 211 | AAAAAGCTCGTAGTTGAACCTTGGGTCTGGC |
| 212 | GACCGATAGCGCACAAGTAGAGT |
| 213 | TGTTGGGACCCGAAAGATGGTGAACTATGCC |
| 214 | CCAACTTAGAACTGGTACGGACAAGGGG |
| 215 | ATTTGTACGGTGAAACTGCGAATGGCTCATTAAATC |
| 216 | GCAAGGCTGAAACTTAAAGAAATTGACGGAAGGGCACC |
| 217 | CGACCAACTTAGAACTGGTACGGACAAGG |
| 218 | CAAATCGATCGTCAAATTTGGGTA |
| 219 | ACAATAAATACTGATACAGGGCTC |
| 220 | TCGCAGCGGTTCTGACGTG |
| 221 | TAGCGACGTTGCTTTTTGATCCT |
| 222 | TGCAGATCTTGGTGGTAGTAGCA |
| 223 | ATTGACAGATTGAGAGCTCTTTCTTG |
| 224 | GCATATCAATAAGCGGAGGAAAAGAAACCAACA |
| 225 | ATAGGGAACGTGAGCTGGG |
| 226 | CAATGTAGATAAGGGAAGTCG |
| 227 | TTTGACCTCGGATCAGGTAGGAGTACCCGCTGAACTTAAGCAT |
| 228 | GTGCAAATCGATCGTCAAATTTGGGTATAGGGGC |
| 229 | TGTTGACGCAATGTGATTTCT |
| 230 | GAACTTTGAGGACTGAAGTGGGGAAAGGTTCCATGTG |
| 231 | AGCATATCAATAAGCGGAGGAAAAGAAACCAACA |
| 232 | AGAACTGGTACGGACAAGGGGA |
| 233 | ATGGAAGTCGGAATCCGCTAAGG |
| 234 | GCAGATCTTGGTGGTAGTAGC |
| 235 | CCGGCATCGATGAAGAACGCAGCG |
| 236 | TTCGATGTCGGCTCTTCCTATC |
| 237 | AAACTTTCAACAACGGATCTCT |
| 238 | AGCGGTTCTGACGTGCAAATC |
| 239 | TGAAACTTAAAGAAATTGACG |
| 240 | TCCGGCATCGATGAAGAACGCAGCG |
| 241 | AAAACTTTCAACAACGGATCTCTT |
| 242 | AGAACTGGTACGGACAAGGGG |
| 243 | TGACAATAAATACTGATACAGGGCTC |
| 244 | TCTAGTTGATAGAACAATGTAGATAAGGGAAGTC |
| 245 | AATCGATCGTCAAATTTGGGTATAGGGGCG |
| 246 | AGTGGTGTTGACGCAATGTGATTTCT |
| 247 | TGACGTGCAAATCGATCGTCAAATTTGGG |
| 248 | GTTGATAGAACAATGTAGATAAGGGAAGTCGGC |
| 249 | GATAGAACAATGTAGATAAGGGAAGTC |
| 250 | TTTGACCTCGGATCAGGTAGGAGTACCCGCTGAA |
| | CTTAAGCATATCAA |
| 251 | TCTCCAAGGTGAAAAGCCTCTAGTTGATAGAACAATG |
| 252 | AGAACTGGTACGGACAAGGGGAATCTGACTG |
| 253 | AAGTCGGAATCCGCTAAGGAGTGTGT |
| 254 | ATCTTGGTGGTAGTAGCAAATACTC |
| 255 | AATGGATCGTTACTAGTGGGCCATTTTTG |
| 256 | GATGCGGGATGAACCGAACGCGAGGTTA |
| 257 | TGACGTGCAAATCGATCGTCAAATTTGGGTA |
| 258 | GTTTGACCTCGGATCAGGTAGGAGTACCC |
| 259 | GTGGCTGTTGGGACCCGAAAGATGGTGAAC |
| 260 | TTGTTGGTTTCTAGGACCGCCGTA |
| 261 | AAAGAAGACCCTGTTGAGCTTGACTCT |
| 262 | GTTTGACCTCGGATCAGGTAGGAGTACCCGC |
| 263 | GAACTGGTACGGACAAGGGGAATCTGACTGTCTAATT |
| 264 | CGGATTATGGCTGAACGCCTCTAAGTC |
| 265 | CCAACTTAGAACTGGTACGGACAAGG |
| 266 | TCAAATGAGAACTTTGAGGACTGAAGTGGG |
| 267 | TTGGTCATTTAGAGGAAGT |
| 268 | TTTGACCTCGGATCAGGTAGGAGTACCCGCTGAACTTA |
| 269 | GTTTGACCTCGGATCAGGTAGGAGTACCCGCTGAACTTAAGC |
| 270 | TCCGTGAATCATCGAATCTTTGAACGCACATT |
| 271 | GACGTTGCTTTTTGATCCTTCGATGTCGGCTCTT |
| 272 | TAGAACTGGTACGGACAAGGGGAATCTGACTG |
| 273 | AACGAACGAGACCTTAACC |
| 274 | GAAGACCCTGTTGAGCTTGACTC |
| 275 | TATTCCGGGGGGCATGCCTGTTCGAGCGTC |
| 276 | AAAGCTCGTAGTTGAACCTTGGGTCTGGC |
| 277 | TTTTTGATCCTTCGATGTCGGCTCTTCC |
| 278 | TTTGACCTCGGATCAGGTAGGAGTACCCGCTGAA |
| | CTTAAGCATATC |

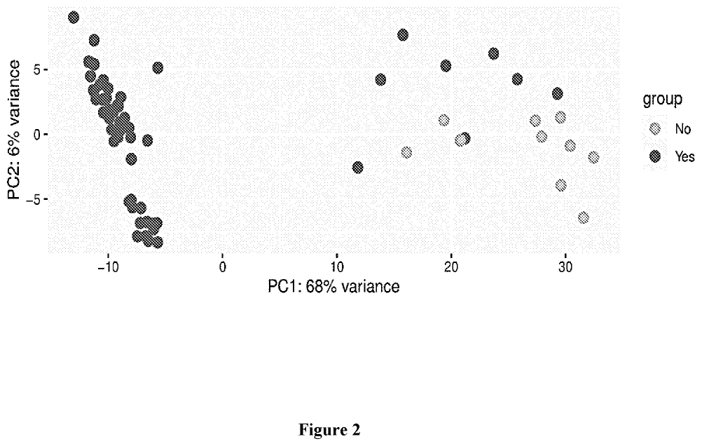

The test using the RiboMarkers identified has a 85% sensitivity rate when compared to clinical diagnosis. 48 out 56 positive patients have significant RiboMarker signature (see FIG. 2).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of detecting a dimorphic fungal infection in a subject comprising detecting at least one small RNA molecule in a sample from the subject associated with the dimorphic fungal infection.

2. The method of claim 1, wherein the at least one small RNA molecule comprises at least 80% identity to one of SEQ ID NO: 1-120.

3. The method of claim 1 or 2, wherein the at least one small RNA molecule is not a ribosomal RNA.

4. The method of any one of claims 1-3, wherein the dimorphic fungal infection is selected from the list consisting of coccidioidomycosis, histoplasmosis, blastomycosis, candidiasis, and a combination thereof.

5. The method of claim 4, wherein the dimorphic fungal infection comprises coccidioidomycosis.

6. The method of claim 4, wherein the dimorphic fungal infection comprises histoplasmosis.

7. The method of claim 4, wherein the dimorphic fungal infection comprises blastomycosis.

8. The method of claim 4, wherein the dimorphic fungal infection comprises candidiasis.

9. The method of any one of claims 1-8, wherein the dimorphic fungal infection results from an infection with a dimorphic fungal organism.

10. The method of claim 9, wherein the dimorphic fungal organism is selected from the list consisting of Coccidioides, Histoplasma capsulatum, Blastomyces, Candida, Lomentospora prolificans, and Scedosporum.

11. The method of claim 10, wherein the dimorphic fungal organism comprises a Coccidioides spp.

12. The method of claim 10, wherein the dimorphic fungal organism comprises Histoplasma capsulatum.

13. The method of claim 10, wherein the dimorphic fungal organism comprises a Blastomyces spp.

14. The method of claim 10, wherein the dimorphic fungal organism comprises a Candida spp.

15. The method of any one of claims 1-14, wherein the at least one small RNA molecule is derived from the dimorphic fungal organism.

16. The method of any one of claims 1-15, wherein the at least one small RNA molecule is no more than 40 nucleotides in size.

17. The method of any one of claims 1-16, wherein detecting the at least one small RNA molecule comprises ligating a single-stranded adapter to the at least one small RNA molecule to produce a adapter-RNA molecule ligation product, wherein the polynucleotides of the plurality of adapter-polynucleotide ligation products comprise a 5′-proximal segment and a 3′-proximal segment, and wherein at least one of the 5′ proximal segment or 3′ proximal segment comprises a sequencing adapter; wherein the adapter comprises:
i. a 5′-proximal segment and a 3′-proximal segment, wherein each proximal segment comprises at least one sequencing adapter, detection sequence, or a combination thereof;
ii. 5′-end and 3′-end groups that allow first and second consecutive ligation reactions either directly or after conversion of one or both of these end groups to ligatable 5′-end and 3′-end group(s); and,
iii. a template-deficient segment that restricts primer extension by a polymerase over the template-deficient segment.

18. The method of claim 17, wherein detecting the at least one small RNA molecule further comprises circularizing the plurality of adapter-polynucleotide ligation products by ligating their 5′ ends to their 3′ ends to produce a plurality of circularized adapter-polynucleotide ligation products.

19. The method of claim 18, wherein detecting the at least one small RNA molecule further comprises hybridizing a first primer comprising a sequence at least partially complementary to the 5′-proximal segment of the adapter, to the circularized adapter-polynucleotide ligation products.

20. The method of claim 19, wherein detecting the at least one small RNA molecule further comprises extending the first primer with a polymerase to produce a plurality of monomeric nucleic acids, wherein each of the monomeric nucleic acids is complementary to: at least one sample polynucleotide of the plurality of sample polynucleotides flanked by at least a portion of the sequencing adapter.

21. The method of claim 20, wherein detecting the at least one small RNA molecule further comprises amplifying the plurality of monomeric nucleic acids using the first primer and a second primer, wherein the sequence of the second primer is at least partially complementary to the 3′-proximal segment of the adapter, to produce amplicon(s) comprising the sequencing library.

22. The method of any one of claims 1-21, wherein the sample is selected from the list consisting of plasma, saliva, serum, and urine.

23. The method of claim 22, wherein the sample comprises plasma.

24. The method of claim 22, wherein the sample comprises saliva.

25. The method of claim 22, wherein the sample comprises serum.

26. The method of claim 22, wherein the sample comprises urine.

27. A method of detecting a dimorphic fungal infection in a subject comprising: detecting at least one nucleic acid comprising any one of SEQ ID NO 1-120 in a sample from a subject.

28. The method of claim 27, comprising determining that the subject is afflicted with the dimorphic fungal infection based on the presence of the at least one nucleic acid.

29. The method of claim 28, comprising determining that the subject is afflicted with a chronic disease associated with the dimorphic fungal infection based on the presence of the at least one nucleic acid.

30. The method of any one of claims 27-29, wherein the method further comprises treating the subject with an antifungal.

31. The method of claim 30, wherein the antifungal is selected from the list consisting of a amphotericin B compound, an azole, and a combination thereof.

32. The method of claim 31, wherein the azole comprises Fluconazole.

33. The method of any one of claim 27-32, wherein detecting the at least one nucleic acid comprises obtaining the sample from a subject.

34. The method of any one of claims 27-33, wherein detecting the at least one nucleic acid comprises isolating at least one nucleic acid from the sample.

35. The method of any one of claims 27-34, wherein detecting the at least one nucleic acid comprises ligating an adapter to the at least one nucleic acid.

36. The method of any one of claims 27-35 wherein detecting the at least one nucleic acid further comprises depleting a second nucleic acid from the sample.

37. The method of claim 36, wherein depleting a second nucleic acid from the sample comprises inhibiting the ligation of a second nucleic acid with an adapter.

38. The method of claim 37, wherein the adapter comprises:
i. a 5′-proximal segment and a 3′-proximal segment, wherein each proximal segment comprises at least one sequencing adapter, detection sequence, or a combination thereof;
ii. 5′-end and 3′-end groups that allow first and second consecutive ligation reactions either directly or after conversion of one or both of these end groups to ligatable 5′-end and 3′-end group(s); and,
iii. a template-deficient segment that restricts primer extension by a polymerase over said template-deficient segment.

39. The method of claim 37, wherein the second nucleic acid comprises at least one sequence selected from SEQ ID NOs 121-278.

40. The method of any one of claims 27-39, comprising detecting at least two nucleic acids comprising any one of SEQ ID NO 1-120 in the sample from the subject.

41. The method of claim 40, comprising detecting at least three amino acids comprising any one of SEQ ID NO 1-120 in the sample from the subject.

42. The method of any one of claims 27-41, wherein the dimorphic fungal disorder is selected from the list consisting of coccidioidomycosis, histoplasmosis, blastomycosis, candidiasis, and a combination thereof.

43. The method of claim 42, wherein the dimorphic fungal disorder comprises coccidioidomycosis.

44. The method of any one of claims 27-43, wherein the dimorphic fungal infection results from an infection with a dimorphic fungal organism.

45. The method of claim 44, wherein the dimorphic fungal organism is selected from the list consisting of Coccidioides, Histoplasma capsulatum, Blastomyces, Candida, Lomentospora prolificans, and Scedosporum.

46. The method of claim 44 or 45, wherein the dimorphic fungal organism comprises a Coccidioides spp.

47. The method of any one claims 27-46, wherein the at least one nucleic acid is derived from a dimorphic fungal organism causing the dimorphic fungal infection.

48. The method of any one of claims 27-47, wherein the at least one nucleic acid comprises an RNA.

49. The method of claim 48, wherein the RNA is no more than 100, 90, 80, 70, 60, 50, 40, 30, 20, or 10 nucleotides in size.

50. The method of claim 49, wherein the RNA is no more than 40 nucleotides in size.

51. The method of any one of the preceding claims, wherein the sample is selected from the list consisting of plasma, saliva, serum, and urine.

52. The method of any one of claims 27-51, wherein the at least one nucleic acid is detected by a nucleic acid amplification reaction.

53. The method of any one of claims 27-52, wherein the at least one nucleic acid is detected by qPCR.

54. The method of any one of claims 27-52, wherein the at least one nucleic acid is detected by next generation sequencing.

55. A method of for preparing a sequencing library for a plurality of sample polynucleotides in a sample from a subject suspected of having a dimorphic fungal disorder, the method comprising:
a) ligating a single-stranded adapter in the form of a plurality of nucleic acid residues to the plurality of sample polynucleotides to produce a plurality of adapter-polynucleotide ligation products, wherein the polynucleotides of the plurality of adapter-polynucleotide ligation products comprise a 5′-proximal segment and a 3′-proximal segment, and wherein at least one of the 5′ proximal segment or 3′ proximal segment comprises a sequencing adapter; wherein the adapter comprises:
i. a 5′-proximal segment and a 3′-proximal segment, wherein each proximal segment comprises at least one sequencing adapter, detection sequence, or a combination thereof;
ii. 5′-end and 3′-end groups that allow first and second consecutive ligation reactions either directly or after conversion of one or both of these end groups to ligatable 5′-end and 3′-end group(s); and,
iii. a template-deficient segment that restricts primer extension by a polymerase over the template-deficient segment;
b) circularizing the plurality of adapter-polynucleotide ligation products by ligating their 5′ ends to their 3′ ends to produce a plurality of circularized adapter-polynucleotide ligation products;
c) hybridizing a first primer comprising a sequence at least partially complementary to the 5′-proximal segment of the adapter, to the circularized adapter-polynucleotide ligation products;
d) extending the first primer with a polymerase to produce a plurality of monomeric nucleic acids, wherein each of the monomeric nucleic acids is complementary to: at least one sample polynucleotide of the plurality of sample polynucleotides flanked by at least a portion of the sequencing adapter; and
e) amplifying the plurality of monomeric nucleic acids using the first primer and a second primer, wherein the sequence of the second primer is at least partially complementary to the 3′-proximal segment of the adapter, to produce amplicon(s) comprising the sequencing library.

56. The method of claim 55, wherein at least one of plurality of nucleic acids comprises at least 80% identity to one of SEQ ID NO: 1-120.

57. The method of claim 55 or 56, wherein the dimorphic fungal disorder is selected from the list consisting of coccidioidomycosis, histoplasmosis, blastomycosis, candidiasis, and a combination thereof.

58. The method of claim 57, wherein the dimorphic fungal disorder comprises coccidioidomycosis.

59. The method of claim 57, wherein the dimorphic fungal disorder comprises histoplasmosis.

60. The method of claim 57, wherein the dimorphic fungal disorder comprises blastomycosis.

61. The method of claim 57, wherein the dimorphic fungal disorder comprises candidiasis.

62. The method of any one of claims 55-61, wherein the dimorphic fungal disorder results from an infection with a dimorphic fungal organism.

63. The method of claim 62, wherein the dimorphic fungal organism is selected from the list consisting of Coccidioides, Histoplasma capsulatum, Blastomyces, Candida, Lomentospora prolificans, and Scedosporum.

64. The method of claim 62 or 63, wherein the dimorphic fungal organism comprises a Coccidioides spp.

65. The method of any one of claims 55-64, wherein the at least one nucleic acid is derived from the dimorphic fungal organism.

66. The method of any one of claims 55-65, wherein the at least one nucleic acid comprises an RNA.

67. The method of claim 66, wherein the RNA is no more than 40 nucleotides in size.

68. The method of any one of claims 55-67, wherein the sample is selected from the list consisting of plasma, saliva, serum, and urine.
